(19) United States
(12) Patent Application Publication
(10) Pub. No.: US 2022/0202674 A1
(43) Pub. Date: Jun. 30, 2022

(54) COSMETIC AND PERSONAL CARE COMPOSITIONS CONTAINING CATIONIC SURFACTANTS AND ANIONIC COMPOUNDS

(71) Applicant: L'OREAL, Paris (FR)

(72) Inventors: XianZhi ZHOU, Millburn, NJ (US); Jun LIANG, Brooklyn, NY (US); Siliu TAN, Westfield, NJ (US)

(73) Assignee: L'OREAL, Paris (FR)

(21) Appl. No.: 17/554,058

(22) Filed: Dec. 17, 2021

Related U.S. Application Data
(60) Provisional application No. 63/131,567, filed on Dec. 29, 2020.

(30) Foreign Application Priority Data
Jun. 10, 2021 (FR) 2106113

(51) Int. Cl. A61K 8/44; A61Q 5/12; A61K 8/41; A61K 8/34; A61K 8/898; A61K 8/92

(52) U.S. Cl. CPC A61K 8/44; A61Q 5/12; A61K 8/416; A61K 2800/5922; A61K 8/898; A61K 8/922; A61K 8/342

(57) ABSTRACT

The instant disclosure relates to cosmetic compositions that include an anionic surfactant having at least two carboxylate salt groups; a cationic surfactant, including a cationizable surfactant; a fatty alcohol; and a cosmetically acceptable solvent; wherein the molar ratio of the cationic surfactant to the anionic surfactant ranges from about 5:1 to about 1:5. Methods for using such cosmetic compositions are also provided.

### CROSS-REFERENCE TO RELATED APPLICATIONS

This application claims benefit of U.S. Provisional Application No. 63/131,567 filed Dec. 29, 2020, and French Patent Application No. 2106113 filed Jun. 10, 2021, which are incorporated herein by reference in their entirety.

### FIELD OF THE DISCLOSURE

The instant disclosure relates to cosmetic and personal care compositions containing cationic surfactants, anionic compounds selected from anionic surfactants, and fatty compounds. The compositions are particularly useful for imparting conditioning or moisturizing properties to keratinous substrates such as hair or skin and have improved rinsability properties. Also disclosed are methods for using the cosmetic compositions.

### BACKGROUND

Many consumers desire to use cosmetic and personal care compositions that provide caring and conditioning/moisturizing properties to keratinous substrates such as hair and skin. For example, consumers continuously seek for skin and scalp care products that can provide improved conditioning and moisturizing benefits. They also seek hair care or hair cosmetic composition products to care for hair that has been subject to chemical treatments such as oxidative hair dyes, hair relaxers, or permanent waving treatments. Many of the chemical treatments as alkaline in nature and may leave residual alkalinity on the treated hair.

While dyeing or color lifting compositions can effectively alter the color of hair, and relaxing, straightening, perming, and waving compositions can effectively alter the shape of the hair, these chemical treatments can damage the hair fibers and/or irritate the scalp. In addition, external factors such as high humidity which causes the hair to become very frizzy, unmanageable, and lose its shape and style or such as mechanical or physical or other external stresses such as brushing, combing, and tangling, and heat can also damage or negatively impact the feel and look of the hair.

Thus, in order to reduce or avoid damage to hair, as well as to improve the cosmetic performance of the compositions, different types of hair products have been developed by manufacturers that are aimed to help consumers improve the quality of hair, feel of the hair, and the manageability of hair as well as reduce or remove the frizziness of hair and condition/moisturize hair, particularly when the hair is damaged. These products are typically provided in forms that are applied as shampoos, conditioners, or treatment such as after or pre-shampooing treatments, and after or pre-conditioning treatments which can be leave-in or rinse-off products.

In addition, consumers desire hair and skin care products, in particular, rinse-off cleansing and rinse-off conditioning products that can be quickly or efficiently removed without leaving noticeable residues and at the same time, deposit conditioning, moisturizing or other cosmetic benefits such as improved hair feel or skin feel properties.

Thus, the object of this invention is related to a composition and method of treating keratinous substrates such as hair or skin wherein the composition will deliver both caring and rinsability/removability properties.

The object of the invention is also to provide a composition and method of treating hair that has been chemically treated or damaged such as hair that has been contacted with a basic or alkaline treatment.

### SUMMARY OF THE DISCLOSURE

It has surprisingly been found that compositions and methods of treating keratinous substrates such as hair or skin according to the present invention provide conditioning and moisturizing benefits to the keratinous substrate, as well as other desirable properties such as fast and efficacious rinsability or removability attributes.

One aspect of the invention pertains to a cosmetic composition containing:
(a) at least one anionic surfactant having at least two carboxylate salt groups:
(b) at least one cationic surfactant, including a cationizable surfactant;
(c) at least one fatty alcohol; and
(d) at least one cosmetically acceptable solvent;
   wherein the mole ratio of the at least one cationic surfactant to the at least one anionic surfactant ranges from about 5:1 to about 1:5.

Another aspect of the invention pertains to methods of treating keratinous substrates such hair or skin, for example, conditioning or moisturizing hair or skin. In some embodiments, the method comprises applying any of the compositions described herein to keratinous substrates. In one or more embodiments, the composition is applied to hair, including curly hair, as part of a hair caring routine. In some embodiments, the composition is applied after treating the hair with a shampoo. In some embodiments, the composition is a rinse-off product. In some embodiments, the rinse-off composition is used as a post-treatment product, following the chemical treatment of hair or treatment of hair with an alkaline product. Such a product can deliver conditioning, moisturizing and repair benefits to the chemically treated hair. In some embodiments, the composition is used as a rinse-off skin care product that can cleanse and at the same time. deliver conditioning and moisturizing benefits.

### BRIEF DESCRIPTION OF THE DRAWING

Implementation of the present technology will now be described, by way of example only, with reference to the attached figure, wherein:

FIG. 1 is a set of graphs showing the viscosity profiles of inventive composition Ex. A when the pH of the composition is changed or when the composition is diluted with water. FIG. 1A is a graph showing the viscosity profiles of inventive composition Ex. A when the pH is changed. FIG. 1B is a graph showing the viscosity profiles of inventive composition Ex. A when the composition is diluted with water.

FIG. 2 is a set of graphs showing the viscosity profiles of inventive composition Ex. B when the pH of the composition is changed or when the composition is diluted with water. FIG. 2A is a graph showing the viscosity profiles of inventive composition Ex. B when the pH is changed. FIG. 2B is a graph showing the viscosity profiles of inventive composition Ex. B when the composition is diluted with water.

FIG. 3 is a set of graphs showing the viscosity profiles of inventive composition Ex. C when the pH of the composition is changed or when the composition is diluted with water. FIG. 3A is a graph showing s the viscosity profiles of inventive composition Ex. C when the pH is changed. FIG. 3B is a graph showing the viscosity profiles of inventive composition Ex. C when the composition is diluted with water.

FIG. 4 is a set of graphs showing the viscosity profiles of comparative composition Ex. D when the pH of the composition is changed or when the composition is diluted with water. FIG. 4A is a graph showing the viscosity profiles of inventive composition Ex. D when the pH is changed. FIG. 4B is a graph showing the viscosity profiles of inventive composition Ex. D when the composition is diluted with water.

FIG. 5 is a set of graphs showing the viscosity profiles of comparative composition Ex. E when the pH of the composition is changed or when the composition is diluted with water. FIG. 5A is a graph showing the viscosity profiles of inventive composition Ex. E when the pH is changed. FIG. 5B is a graph showing the viscosity profiles of inventive composition Ex. E when the composition is diluted with water.

FIG. 6 is a set of graphs showing the viscosity profiles of comparative composition Ex. F when the pH of the composition is changed or when the composition is diluted with water. FIG. 6A is a graph showing the viscosity profiles of inventive composition Ex. F when the pH is changed. FIG. 6B is a graph showing the viscosity profiles of inventive composition Ex. F when the composition is diluted with water.

FIG. 7 is a set of graphs showing the viscosity profiles of comparative composition Ex. G when the pH of the composition is changed or when the composition is diluted with water. FIG. 7A is a graph showing the viscosity profiles of inventive composition Ex. G when the pH is changed. FIG. 7B is a graph showing the viscosity profiles of inventive composition Ex. G when the composition is diluted with water.

FIG. 8 is a set of graphs showing the viscosity profiles of comparative composition Ex. H when the pH of the composition is changed or when the composition is diluted with water. FIG. 8A is a graph showing the viscosity profiles of inventive composition Ex. H when the pH is changed. FIG. 8B is a graph showing the viscosity profiles of inventive composition Ex. H when the composition is diluted with water.

It should be understood that the various aspects are not limited to the arrangements and instrumentality shown in the drawings.

### DETAILED DESCRIPTION OF THE DISCLOSURE

The term “cosmetic composition” encompasses many types of compositions for application to keratinous substrates such as hair or skin. When the substrate is hair, the compositions can take be hair lotions, hair emulsion creams, hair conditioners, hair masques (masks), etc., which can be used either as leave-on or rinse-off treatments or products. A cosmetic composition such as a hair cosmetic composition according to the invention is characterized by its ability to provide a cosmetic (such as caring) benefit to the substrate. Non-limiting examples of benefits that can be imparted by the compositions of the present invention to hair include one or more of frizz control, curl definition, discipline, volume control, manageability, smoothness, sleekness, softness, suppleness, hydration or moisture (does not feel dry) and natural feel. At the same time, even when the compositions of the present disclosure contain fatty compounds such as fatty alcohols, silicones, and plant- or vegetable-based oils, surprisingly, a light weight feel and a clean feel (non-greasy, non-oily) are imparted to the hair.

The cosmetic compositions of the instant disclosure typically include:
(a) at least one anionic surfactant having at least two carboxylate salt groups;
(b) at least one cationic surfactant, including a cationizable surfactant;
(c) at least one fatty alcohol; and
(d) at least one cosmetically acceptable solvent;
   wherein the mole ratio of the at least one cationic surfactant to the at least one anionic surfactant ranges from about 5:1 to about 1:5.

In an embodiment, the cosmetic composition has a lamellar gel network.

In an embodiment, the at least one anionic surfactant having at least two carboxylate salt groups is represented by the formula (I):

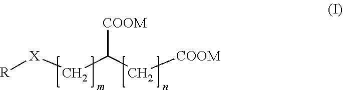

wherein:
R represents a saturated or unsaturated, linear or branched hydrocarbon group having 8 to 30 carbon atoms,
X is —O— or an ester group, —COO—, or amide group, —CONY wherein Y is hydrogen or an alkyl group,
m is 0 or 1 or 2,
n is an integer of 0 to 4,
M is hydrogen or a salt-forming cation wherein COO is the counter-anion, such as for example sodium, potassium, ammonium, or triethanolamine.

In an embodiment, the at least one anionic surfactant having at least two carboxylate salt groups is represented by the formula (I):

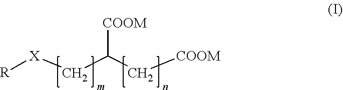

wherein:

R represents a saturated or unsaturated, linear or branched hydrocarbon group having 10 to 22 carbon atoms,
X is an amide group, —CONY wherein Y is hydrogen or an alkyl group, preferably, hydrogen,
m is 0 or 1 or 2,
n is an integer of 0 to 4,
M is hydrogen or a salt-forming cation wherein COO is the counter-anion, such as for example sodium, potassium, ammonium, or triethanolamine.

In an embodiment, the at least one anionic surfactant having at least two carboxylate salt groups is selected from sodium stearoyl glutamate, disodium stearoyl glutamate, sodium cocoyl glutamate, disodium cocoyl glutamate, sodium lauroyl glutamate, or a mixture thereof.

In an embodiment, the at least one anionic surfactant having at least two carboxylate salt groups is present in an amount of from about 0.5 to about 5 wt. %, or about 0.5 to about 4 wt. %, or about 0.6 to about 3.5 wt. %, or about 0.6 to about 3 wt. %, or about 0.7 to about 2.5 wt. %, or about 0.8 to about 2 wt. %, or about 1 to about 2 wt. %, or about 1.2 to about 2 wt. %, based on the total weight of the cosmetic composition, including ranges and sub-ranges there between.

In an embodiment, the at least one cationic surfactant is selected from:
quaternary ammonium salts corresponding to the general formula below:

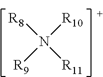

in which the groups R₆ to R₁₁, which may be identical or different, represent a linear or branched, saturated or unsaturated aliphatic group comprising from 1 to 30 carbon atoms, or an aromatic group such as aryl or alkylaryl, at least one of the groups R₈ to R₁₁ denoting a group comprising from 8 to 30 carbon atoms;
a quaternary ammonium salt of imidazoline;
a quaternary diammonium or triammonium salt, in particular of formula:

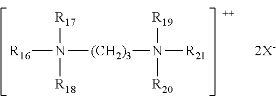

in which R₁₆ denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ)(R₁₇ₐ)(R₁₈ₐ)N—(CH₂)₃, R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms, and X⁻ is an anion chosen from the group of halides, acetates, phosphates, nitrates and methyl sulfates; and
cationizable surfactants, including cationizable surfactants together with an acid neutralizer selected from compounds of the general structure R₄-A-R₅—B,
wherein R₄ is a saturated or unsaturated, straight or branched alkyl chain with 8 to 24 C atoms, R₅ is a straight or branched alkyl chain with 1 to 4 C atoms, A is selected from:

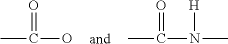

and B is selected from

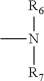

wherein R₆ and R₇ are the same or different is H or an alkyl chain with 1 to 4 C atoms, hydroxyl alkyl chain with 1 to 4 C atoms and di hydroxyl alkyl chain with 2 to 4 C atoms, and

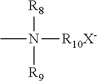

wherein R₈ and R₉ are the same or different, an alkyl chain with 1 to 4 C atoms, hydroxyl alkyl chain with 1 to 4 C atoms and di hydroxyl alkyl chain with 2 to 4 C atoms, R₁₀ is an alkyl chain with 1 to 4 C atoms, hydroxyl alkyl chain with 1 to 4 C atoms or di hydroxyl alkyl chain with 2 to 4 C atoms,
or mixtures thereof.

In an embodiment, the at least one cationic surfactant is selected from quaternary diammonium or triammonium salts, in particular of formula:

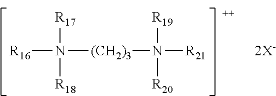

in which R₁₆ denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ)(R₁₇ₐ)(R₁₈ₐ)N—(CH₂)₃, R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms, and X⁻ is an anion chosen from the group of halides, acetates, phosphates, nitrates and methyl sulfates.

In an embodiment, the at least one cationic surfactant is selected from cationizable surfactants, including cationizable surfactants together with an acid neutralizer selected from compounds of the general structure R₄-A-R₅—B,
wherein R₄ is a saturated or unsaturated, straight or branched alkyl chain with 8 to 24 C atoms, R₅ is a straight or branched alkyl chain with 1 to 4 C atoms, A is selected from:

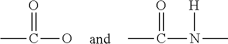

and B is selected from

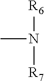

wherein R₆ and R₇ are the same or different is H or an alkyl chain with 1 to 4 C atoms, hydroxyl alkyl chain with 1 to 4 C atoms and di hydroxyl alkyl chain with 2 to 4 C atoms, and

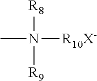

wherein R₈ and R₉ are the same or different, an alkyl chain with 1 to 4 C atoms, hydroxyl alkyl chain with 1 to 4 C atoms and di hydroxyl alkyl chain with 2 to 4 C atoms, R₁₀ is an alkyl chain with 1 to 4 C atoms, hydroxyl alkyl chain with 1 to 4 C atoms or di hydroxyl alkyl chain with 2 to 4 C atoms.

In an embodiment, the at least one cationic surfactant is selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and mixtures thereof.

In an embodiment, the at least one cationic surfactant includes behentrimonium chloride.

In an embodiment, the at least one cationic surfactant includes cetrimonium chloride.

In an embodiment, the at least one cationic surfactant is chosen from behentrimonium chloride, cetrimonium chloride, or a mixture thereof.

In an embodiment, the at least one cationic surfactant is selected from oleamidopropyl dimethylamine, linoleamidopropyl dimethylamine, isostearamidopropyl dimethylamine, oleyl hydroxyethyl imidazoline, stearamidopropyl dimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and mixtures thereof.

In an embodiment, the at least one cationic surfactant includes stearamidopropyl dimethylamine.

In an embodiment, the at least one cationic surfactant is present in an amount of from about 0.1 to about 5 wt. %, or about 0.1 to about 4 wt. %, or about 0.2 to about 4 wt. %, or about 0.3 to about 3 wt. %, or about 0.5 to about 2.5 wt. %, based on the total weight of the cosmetic composition, including ranges and sub-ranges there between.

In an embodiment, the at least one fatty alcohol is selected from cetyl alcohol, stearyl alcohol, cetearyl alcohol (combination of cetyl alcohol and stearyl alcohol), behenyl alcohol, lauryl alcohol (1-dodecanol); myristic or myristyl alcohol (1-tetradecanol), arachidyl alcohol (1-eicosanol), lignoceryl alcohol (1-tetracosanol); ceryl alcohol (1-hexacosanol); montanyl alcohol (1-octacosanol); myricylic alcohol (1-triacontanol), decyl alcohol, undecyl alcohol, and a mixture thereof.

In an embodiment, the at least one fatty alcohol is present in an amount of from about 0.5 to about 10 wt. %, or from about 1 to about 8 wt. %, or from about 1.5 to about 7 wt. %, or from about 2 to about 7 wt. %, or from about 2 to about 6 wt. %, based on the total weight of the cosmetic composition, including ranges and sub-ranges there between.

In an embodiment, the at least one cosmetically acceptable solvent is selected from water, organic solvents, and mixtures thereof.

In an embodiment, the molar ratio of the at least one cationic surfactant to the at least one anionic surfactant having at least two carboxylate salt groups is greater than 1.

In an embodiment, the molar ratio of the at least one cationic surfactant to the at least one anionic surfactant having at least two carboxylate salt groups ranges from about 1:0.9 to about 1:0.2, or from about 1:0.8 to about 1:0.4 or from about 1:0.7 to about 1:0.5, including ranges and sub-ranges there between.

In an embodiment, the molar ratio of the at least one cationic surfactant to the at least one anionic surfactant is less than 1.

In an embodiment, the molar ratio of the at least one cationic surfactant to the at least one anionic surfactant having at least two carboxylate salt groups ranges from about 1:2 to about 1:1.1, or from about 1:1.8 to about 1:1.2 or from about 1:1.6 to about 1:1.2, including ranges and sub-ranges there between.

In an embodiment, the molar ratio of the at least one cationic surfactant to the at least one anionic surfactant is 1.

In an embodiment, the cosmetic composition has a pH ranging from about 2.5 to less than about 5, or from about 3 to about 4.8, or from about 3 to about 4.5, or from about 3.2 to about 4.5, or from about 3 to about 4, including ranges and sub-ranges there between.

In an embodiment, the cosmetic composition of the present disclosure has an initial viscosity (after preparation of the composition) of equal to or greater than 1 Pa·s, or from about 1 to about 100 Pa·s, or about 1 to about 50 Pa·s, or about 1 to about 30 Pa·s, or about 1 to about 20 Pa·s, including ranges and sub-ranges there between, at a shear rate at about or above 10 (1/s) and below 10² 1/s as measured a rheometer (DHR-2, TA instruments, New Castle, Del., USA).

In an embodiment, the molar ratio of the at least one cationic surfactant to the at least one anionic surfactant having at least two carboxylate salt groups is greater than about 1, and the initial pH of the composition is less than 5 (such as from about 3 to about 4.8).

In an embodiment, the molar ratio of the at least one cationic surfactant to the at least one anionic surfactant having at least two carboxylate salt groups is less than 1, and the initial pH of the composition ranges from greater than about 3.5 to less than about 5, or from about 4 to less than about 5 (such as at about 4.8).

In an embodiment, the composition of the present invention further comprises at least one amino functionalized silicone selected from amodimethicone, bis-hydroxy/methoxy amodimethicones, bis-cetearyl amodimethicone, amodimethicone, bis(C13-15 alkoxy) PG amodimethicones, aminopropyl phenyl trimethicones, aminopropyl dimethicones, bis-amino PEG/PPG-41/3 aminoethyl PG-propyl dimethicones, caprylyl methicones, and a mixture thereof.

In an embodiment, the at least one amino functionalized silicone is present in an amount of about 0.1 to about 5 wt. %, or preferably about 0.3 to about 4 wt. %, or more preferably, about 0.4 to about 3 wt. %, or about 0.5 to about 2 wt. %, based on the total weight of the cosmetic composition, including ranges and sub-ranges there between.

In an embodiment, the composition of the present invention further comprises at least one fatty compound selected from at least one fatty compound is selected from plant-based oils (such as plant-based butters, plant-based triglycerides), hydrocarbon oils, esters, or a mixture thereof.

In an embodiment, the at least one fatty compound is present in an amount of about 0.1 to about 10 wt %, about 0.1 to about 9 wt. %, or about 0.2 to about 8 wt. %, or about 0.3 to about 7 wt. %, or about 0.4 to about 7 wt. %, or about 0.4 to about 6.5 wt. %, or about 0.5 to about 6 wt. %, or 0.5 to about 5 wt. %, or about 0.5 to about 4.5 wt. %, or preferably, about 1 to about 4 wt. %, or more preferably, about 1 to about 3 wt. %, or even more preferably, about 1.5 to about 2.5 wt %, based on the total weight of the cosmetic composition, including ranges and sub-ranges there between.

In an embodiment, the least one fatty compound includes at least one ester selected from fatty esters, cetyl esters, isopropyl esters, glyceryl (glycerol) esters, dialkyl esters, diesters with octanoic acid and propylene glycol (for example, mixture of the propylene glycol diesters of caprylic and capric acids, propylene glycol dicaprylate/dicaprate, or mixtures thereof, preferably, from cetyl esters, isopropyl esters, glyceryl esters, or mixtures thereof.

In an embodiment, the at least one ester is present in an amount of about 0.01 to about 6 wt %, such as about 0.05 to about 5 wt. %, about 0.05 to about 4.5 wt. %, or such as about 0.1 to about 4 wt. %, based on the total weight of the cosmetic composition, including ranges and sub-ranges there between.

In an embodiment, the total amount of the at least one fatty alcohol(s) is greater than the total amount of the at least one fatty compound in the compositions of the present invention, and ranges from about 10:1 to about 1.5:1, including ranges and sub-ranges there between. In an embodiment, the weight ratio of the at least one fatty alcohol(s) is greater than the total amount of the at least one fatty compound is greater than 1, or is at about 9:1, 8:1, 7:1, 6; 1, 5.7:1, 5.5:1, 5:1, 4:1, 3:1, 2:1, or 1.5:1.

In an embodiment, the composition of the present invention further comprises at least one nonionic surfactant selected from alkoxylated fatty alcohols, alkylpolyglucosides, polysorbates, or mixtures thereof.

The cosmetically acceptable solvent is selected from water, organic solvents, or a mixture thereof.

In an embodiment, the cosmetically acceptable solvent comprises water.

In an embodiment, the cosmetically acceptable solvent comprises water and at least one organic solvent.

In an embodiment, the cosmetic composition of the present invention typically includes:
(a) from about 0.5 to about 5 wt. %, or about 0.5 to about 4 wt. %, or about 0.6 to about 3.5 wt. %, or about 0.6 to about 3 wt. %, or about 0.7 to about 2.5 wt. %, of at least one anionic surfactant having at least two carboxylate salt groups;
(b) from about 0.2 to about 4 wt. %, or about 0.3 to about 3 wt. %, or about 0.5 to about 2.5 wt. %, of at least one cationic surfactant, including a cationizable surfactant;
(c) from about 1.5 to about 7 wt. %, or from about 2 to about 7 wt. %, or from about 2 to about 6 wt. %, of at least one fatty alcohol;
(d) at least one cosmetically acceptable solvent;
(e) optionally, from about 0.1 to about 2 wt. % of at least one silicone;
(f) optionally, from about 0.05 to about 2 wt. % of at least one nonionic surfactant; and
(g) optionally, from about 0.1 to about 5 wt. % of at least one fatty compound selected from plant-based oils, hydrocarbon oils, esters, fatty acids, or a mixture thereof; and
wherein the weight ratio of the at least one cationic surfactant to the at least one anionic surfactant ranges from about 3:1 to about 1:3 or from about 2:1 to about 1:2;
wherein the cosmetic composition has a pH ranging from about 2.5 to less than about 5, or from about 3 to about 4.8, or from about 3 to about 4.5, or from about 3.2 to about 4.5;
all weights being based on the total weight of the cosmetic composition.

In an embodiment, the cosmetic composition of the present invention typically includes:
(a) from about 0.7 to about 2.5 wt. % of at least one anionic surfactant having at least two carboxylate salt groups selected from sodium stearoyl glutamate, disodium stearoyl glutamate, or a mixture thereof;
(b) from about 0.5 to about 2.5 wt. % of at least one cationic surfactant, including a cationizable surfactant and selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, or mixtures thereof;
(c) from about 2 to about 6 wt. % of at least one fatty alcohol selected from cetyl alcohol, stearyl alcohol, cetearyl alcohol (combination of cetyl alcohol and stearyl alcohol), behenyl alcohol, lauryl alcohol (1-dodecanol); myristic or myristyl alcohol (1-tetradecanol), decyl alcohol, undecyl alcohol, and a mixture thereof;
(d) at least one cosmetically acceptable solvent;
(e) optionally, from about 0.1 to about 2 wt. % of at least one silicone;
(f) optionally, from about 0.05 to about 2 wt. % of at least one nonionic surfactant; and
(g) optionally, from about 0.1 to about 5 wt. % of at least one fatty compound selected from plant-based oils, hydrocarbon oils, esters, fatty acids, or a mixture thereof; and
wherein the weight ratio of the at least one cationic surfactant to the at least one anionic surfactant ranges from about 1:2 to about 1:1.1;

all weights being based on the total weight of the cosmetic composition.

In an embodiment, the cosmetic composition of the present invention typically includes:
(a) from about 1.2 to about 2 wt. % of at least one anionic surfactant having at least two carboxylate salt groups selected from sodium stearoyl glutamate, disodium stearoyl glutamate, or a mixture thereof;
(b) from about 0.5 to about 2.5 wt. % of at least one cationic surfactant, including a cationizable surfactant;
(c) from about 2 to about 6 wt. % of at least one fatty alcohol selected;
(d) at least one cosmetically acceptable solvent;
(e) optionally, from about 0.1 to about 2 wt. % of at least one silicone;
(f) optionally, from about 0.05 to about 2 wt. % of at least one nonionic surfactant; and
(g) optionally, from about 0.1 to about 5 wt. % of at least one fatty compound selected from plant-based oils, hydrocarbon oils, esters, fatty acids, or a mixture thereof; and
wherein the weight ratio of the at least one cationic surfactant to the at least one anionic surfactant ranges from about 1:0.7 to about 1:0.5;

all weights being based on the total weight of the cosmetic composition.

In an embodiment, the present disclosure is directed to a method comprising applying onto hair, any one of the above-described cosmetic compositions wherein the method imparts to hair, one or more of:
conditioning benefits;
hair repair benefits;
frizz control;
manageability;
curl definition;
sleek look;
smoothness; or
softness.

In an embodiment, the above-described method comprises or includes a step of alkalizing hair or forming pre-alkalized hair before applying the cosmetic composition onto the hair.

In an embodiment, the present invention is further directed to a fast wash or a fast rinse method, the method comprising:
(1) applying onto a keratinous substrate such as hair or skin, a cosmetic composition comprising:
   (a) from about 0.5 to about 4 wt. %, or about 0.6 to about 3.5 wt. %, or about 0.6 to about 3 wt. %, or about 0.7 to about 2.5 wt. %, or about 0.8 to about 2 wt. %, or about 1 to about 2 wt. %, or about 1.2 to about 2 wt. %, of at least one anionic surfactant having at least two carboxylate salt groups;
   (b) from about 0.2 to about 4 wt. %, or about 0.3 to about 3 wt. %, or about 0.5 to about 3 wt. %, of at least one cationic surfactant, including a cationizable surfactant;
   (c) from about 2 to about 7 wt. %, or from about 3 to about 7 wt. %, or about 4 to about 7 wt. %, of at least one fatty alcohol;
   (d) at least one cosmetically acceptable solvent;
   (e) optionally, at least one silicone;
   (f) optionally, at least one nonionic surfactant; and
   (g) optionally, at least one fatty compound selected from non-silicone oils, esters, fatty acids, or a mixture thereof; and
      wherein the mole ratio of the at least one cationic surfactant to the at least one anionic surfactant ranges from about 5:1 to about 1:5; and
      all weights being based on the total weight of the cosmetic composition.
      In an embodiment, the method further comprises a step of rinsing the keratinous substrate with extraneous water.

It has been surprisingly discovered that the combination of the least one anionic surfactant having at least two carboxylate salt groups and of the at least one cationic surfactant in a molar ratio greater than 1 in a composition for treating hair or skin, resulted in a faster and improved ease of rinsability of the composition when it is applied onto wet hair or skin or exposed to extraneous water. When exposed to extraneous water, the viscosity of the composition significantly decreases. With a faster and improved ease of rinsability, the rinsing step uses less water.

In various embodiments, the molar ratio of the at least one anionic surfactant having at least two carboxylate salt groups to the at least one cationic surfactant ranges from about 1:0.9 to about 1:0.2, or from about 1:0.8 to about 1:0.4 or from about 1:0.7 to about 1:0.5, including ranges and sub-ranges there between, or is at about 1:0.9, 1:0.85, 1:0.8, 1:0.75, 1:07, 1:0.65, 1:0.6, 1:0.55, 1:0.5, 1:0.45, 1:0.4, 1:0.35, 1:0.3, 1:0.25, or 1:0.2.

Thus, in an embodiment, the present invention is further directed to a fast wash or a fast rinse method, the method being a method of improving the rinsability of a composition from keratinous substrates such as hair of skin and comprising:
(1) combining:
   (a) from about 0.5 to about 4 wt. %, or about 0.6 to about 4 wt. %, or about 0.6 to about 3 wt. %, or about 0.7 to about 2.5 wt. %, of at least one anionic surfactant having at least two carboxylate salt groups;
   (b) from about 0.2 to about 4 wt. %, or about 0.3 to about 3 wt. %, or about 0.5 to about 3 wt. %, of at least one cationic surfactant, including a cationizable surfactant;
   (c) from about 2 to about 7 wt. %, or from about 3 to about 7 wt. %, or about 4 to about 7 wt. % of at least one fatty alcohol;
   (d) at least one cosmetically acceptable solvent;
   (e) optionally, from about 0.1 to about 2 wt. %, of at least one silicone;
   (f) optionally, from about 0.05 to about 2 wt. %, of at least one nonionic surfactant; and
   (g) optionally, from about 0.1 to about 5 wt. %, of at least one fatty compound selected from non-silicone oils, esters, fatty acids, or a mixture thereof;

wherein the molar ratio of the at least one cationic surfactant to the at least one anionic surfactant is greater than 1, in order to form a cosmetic composition; and

all weights being based on the total weight of the cosmetic composition.

When the cosmetic composition is contacted with extraneous water, the viscosity of the composition decreases which allows for improved spreadability of the composition on skin or hair.

In various embodiments, when the cosmetic composition of the present disclosure is to be applied onto hair, any one of the above-described methods includes applying the cosmetic composition onto hair that has been previously contacted or pre-treated with an alkaline or basic product or composition, including an oxidative hair dyeing composition or a hair relaxing/straightening composition.

It has also been surprisingly discovered that the combination of the least one anionic surfactant having at least two carboxylate salt groups and of the at least one cationic surfactant in a molar ratio greater than 1 in a composition for treating hair or, resulted in a faster and improved ease of rinsability of the composition when the pH of the composition is increased, such as when the composition is put in contact with an alkaline or basic composition or substrate or when the composition is applied onto pre-alkalized hair. When the pH of the composition increases, viscosity of the composition significantly decreases. With a faster and improved ease of rinsability, the rinsing step uses less water.

Thus, in an embodiment, the present invention is further directed to a fast wash or a fast rinse method, the method comprising applying onto pre-alkalized hair or hair having a basic or alkaline pH, a cosmetic composition comprising:
(a) at least one anionic surfactant having at least two carboxylate salt groups;
(b) at least one cationic surfactant, including a cationizable surfactant;
(c) at least one fatty alcohol;
(d) at least one cosmetically acceptable solvent;
(e) optionally, at least one silicone;
(f) optionally, at least one nonionic surfactant; and
(g) optionally, at least one fatty compound selected from non-silicone oils, esters, fatty acids, or a mixture thereof; and

wherein the molar ratio of the at least one cationic surfactant to the at least one anionic surfactant is greater than 1. In an embodiment, the combination of the least one anionic surfactant having at least two carboxylate salt groups and of the at least one cationic surfactant in a molar ratio of less than 1 in a composition for treating hair or skin, can result in better deposition of conditioning and/or active agents on hair or skin when the pH of the composition was increased such as when the composition is put in contact with or applied onto pre-alkalized hair. When the pH of the composition increases, the viscosity of the composition significantly increases.

In various embodiments, the molar ratio of the at least one anionic surfactant having at least two carboxylate salt groups to the at least one cationic surfactant ranges from about 1:2 to about 1:1.1, or from about 1:1.8 to about 1:1.2 or from about 1:1.6 to about 1:1.2, including ranges and sub-ranges there between, or is at about 1:2, 1:1.9, 1:1.8, 1:1.7, 1:1.6, 1:1.5, 1:1.4, 1:1.3, 1:1.2, or 1:1.1.

Thus, in an embodiment, the present invention is further directed to a method of improving the deposition of conditioning and/or active agents on keratinous substrates, the method comprising:
(1) applying onto a keratinous substrate such as hair or skin, a cosmetic composition comprising:
   (a) at least one anionic surfactant having at least two carboxylate salt groups;
   (b) at least one cationic surfactant, including a cationizable surfactant;
   (c) at least one fatty alcohol;
   (d) at least one cosmetically acceptable solvent;
   (e) optionally, at least one silicone;
   (f) optionally, at least one fatty compound selected from non-silicone oils, esters, fatty acids, or a mixture thereof; and
      wherein the molar ratio of the at least one cationic surfactant to the at least one anionic surfactant is less than 1.

In an embodiment, any one of the above-described methods further comprises a step of rinsing the keratinous substrate with extraneous water.

The cosmetic compositions described herein may be in any suitable physical form. Suitable forms include, but are not limited to low to moderate viscosity liquids, lotions, milks, gel creams, emulsion creams, pastes, clays, conditioners, masks, and the like. In an embodiment, the cosmetic compositions are rinse-off compositions.

The cosmetic compositions may be packaged in a variety of different containers, such as, for example, a ready-to-use container. Non-limiting examples of useful packaging include tubes, jars, caps, unit dose packages, and bottles, including squeezable tubes and bottles, pump bottles, and spray bottles.

### Anionic Surfactant

The anionic surfactant of the present invention has at least two carboxylate salt groups.

In an embodiment, the anionic surfactant having at least two carboxylate salt groups of the present invention can also be described as an amino acid surfactant is an acyl amino acid salt derived from a carboxylate salt of an amino acid wherein the amine group situated on the a-carbon or beta-carbon of an amino acid salt is acylated with an alkyl fatty acid derivative such as a C₈ to C₃₀ fatty acid derivative, or a C₁₀ to C₂₂ fatty acid derivative. The acyl amino acid salts can be formed by conventional means such as by neutralization of the respective amino acid with a base. The amine group situated on the a-carbon or beta-carbon of the neutralized amino acid is acylated with a fatty acid halide (acyl halide) in the presence of a base via the well-known Schotten-Baumann reaction giving the amide, thus forming the desired surfactant reaction product, i.e. the amino acid surfactant. Suitable acyl halides for acylation of the amino acid carboxylate salt include acyl chlorides, bromides, fluorides, and iodides. The acyl halides can be prepared by reacting a saturated or unsaturated, linear or branched C₈ to C₃₀ fatty acid with a thionyl halide (bromide, chloride, fluoride, and iodide). Representative acyl halides include but are not limited to the acyl chlorides selected from decanoyl chloride, dodecanoyl chloride (lauroyl chloride), cocoyl chloride (coconut oil derived fatty acid chlorides) tetradecanoyl chloride (myristoyl chloride), hexadecanoyl chloride (palmitoyl chloride), octadecanoyl chloride (stearoyl chloride), 9-octadecenoyl chloride (oleoyl chloride), eicosanoyl chloride (arachidoyl chloride), docosanoyl chloride (behenoyl chloride), and any mixture thereof. Other acyl halides include the bromides, fluorides and iodides of the foregoing fatty acids. A method for preparing acyl halides as well as an alternative method for acylating amino acids is set forth in US Patent Application Publication No. 2008/0200704, published on Aug. 21, 2008, which application is incorporated herein by reference.

In one embodiment, said anionic surfactant having at least two carboxylate salt groups is represented by the formula (I):

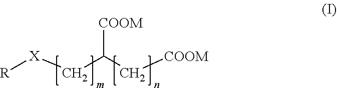

wherein:
R represents a saturated or unsaturated, linear or branched hydrocarbon group having 8 to 30 carbon atoms,
X is —O— or an ester group, —COO—, or amide group, —CONY wherein Y is hydrogen or an alkyl group,
m is 0 or 1 or 2,
   n is an integer of 0 to 4,
   M is hydrogen or a salt-forming cation wherein COO is the counter-anion, such as for example sodium, potassium, ammonium, or triethanolamine.

In an embodiment, in formula (I):
R represents a saturated or unsaturated, linear or branched hydrocarbon group having 10 to 22 carbon atoms,
X is an amide group, —CONY wherein Y is hydrogen or an alkyl group,
m is 0 or 1 or 2,
n is an integer of 0 to 4,
   M is hydrogen or a salt-forming cation wherein COO is the counter-anion, such as for example sodium, potassium, ammonium, or triethanolamine.

In one embodiment, in formula (I):
R represents a saturated or unsaturated, linear or branched hydrocarbon group having 10 to 22 carbon atoms,
X is an amide group, —CONY wherein Y is hydrogen,
m is 0 or 1 or 2,
n is an integer of 0 to 4,
   M is hydrogen or a salt-forming cation wherein COO is the counter-anion, such as for example sodium, potassium, ammonium, or triethanolamine.

In an embodiment, in formula (I):
R represents a saturated or unsaturated, linear or branched hydrocarbon group having 10 to 22 carbon atoms,
X is an amide group, —CONY wherein Y is hydrogen,
m is 0,
n is an integer of 2,
   M is hydrogen or a salt-forming cation wherein COO is the counter-anion, such as for example sodium, potassium, ammonium, or triethanolamine.

Examples of the at least one anionic surfactant having at least two carboxylate salt groups include salts of aspartic acid, glutamic acid, and any mixture thereof. More specific examples are dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, sodium lauroyl aspartate, sodium myristoyl aspartate, sodium cocoyl aspartate, sodium caproyl aspartate, disodium lauroyl aspartate, disodium myristoyl aspartate, disodium cocoyl aspartate, disodium caproyl aspartate, potassium lauroyl aspartate, potassium myristoyl aspartate, potassium cocoyl aspartate, potassium caproyl aspartate, dipotassium lauroyl aspartate, dipotassium myristoyl aspartate, dipotassium cocoyl aspartate, dipotassium caproyl aspartate, and mixtures thereof.

References can be made to the commercially available anionic surfactants, for example, N-acylglutamates, for instance the triethanolamine monococoylglutamate sold under the name Acylglutamate CT-12(R) by the company Ajinomoto and the triethanolamine lauroylglutamate sold under the name ACYLGLUTAMATE LT-12 by the company Ajinomoto; aspartates, for instance the mixture of triethanolamine N-lauroyl aspartate and of triethanolamine N-myristoylaspartate, sold under the name ASPARACK by the company Mitsubishi; citrates, and any mixture thereof.

According to the present invention, the preferred anionic surfactant is selected from, sodium cocoyl glutamate, disodium cocoyl glutamate, sodium lauroyl glutamate, sodium stearoyl glutamate, sodium lauroyl aspartate or a mixture thereof.

Mentions of the preferred anionic surfactants in the composition which is commercially available can be made to sodium cocoyl glutamate (and) disodium cocoyl glutamate (AMISOFT CS-22 sold by Ajinomoto), and sodium lauroyl glutamate (AMISOFT LS-11 sold by Ajinomoto).

Mention may also be made of triethanolamine cocoyl glutamate sold under the name AMISOFT CT12 by the company Ajinomoto, and triethanolamine lauroyl glutamate sold under the name Acylglutamate LT-12 by the company Ajinomoto.

As acyl glutamic acid salt, mention may also be made of sodium hydrogenated tallowoyl glutamate, such as the product sold under the reference Acylglutamate HS 11 by the company Ajinomoto and disodium hydrogenated tallow glutamate, such as the product sold under the reference Acylglutamate HS-21 by the company Ajinomoto.

Mention may also be made of commercial mixtures of surfactants comprising at least one glutamic acid derivative or a salt of said derivative, for instance the mixture of acyl glutamate salts such as AMISOFT LS-22 sold by Ajinomoto.

More preferentially, the anionic surfactant in the compositions of the present invention is sodium stearoyl glutamate (INCI name).

Such compounds are sold under the name AMISOFT by the company Ajinomoto and in particular under the references AMISOFT CA, AMISOFT LA, AMISOFT HS 11 PF, AMISOFT MK-11, AMISOFT LK-11 and AMISOFT CK-11, or alternatively under the name EUMULGIN SG by the company Cognis.

According to one preferred embodiment of the invention, the monosodium salt of n-stearoyl-L-glutamic acid (INCI name: sodium stearoyl glutamate), such as the product sold by the company Ajinomoto under the reference AMISOFT HS 11 PF, is used.

The total amount of the at least one anionic surfactant having at least two carboxylate salt groups in the composition, if present, may vary but is typically from greater than 0.1 to about 5 wt. %, based on the total weight of the composition. In some instances, the total amount of the at least one anionic surfactant having at least two carboxylate salt groups is in an amount of about 0.5 to about 5 wt. %, or about 0.5 to about 4 wt. %, or about 0.6 to about 3.5 wt. %, or about 0.6 to about 3 wt. %, or about 0.7 to about 2.5 wt. %, or about 0.8 to about 2 wt. %, or about 1 to about 2 wt. %, or about 1.2 to about 2 wt. %, based on the total weight of the composition, including ranges and sub-ranges there between.

Thus, the at least one anionic surfactant having at least two carboxylate salt groups is present, by weight, based on the total weight of the composition, in an amount from about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, to about 2 wt. %, including increments and ranges therein and there between.

### Cationic Surfactants Including Cationizable Surfactants

In accordance with the disclosure, compositions hereof may include at least one cationic surfactant. The term “cationic surfactant” means a surfactant that may be positively charged when it is contained in the compositions according to the disclosure. This surfactant may bear one or more positive permanent charges or may contain one or more functional groups that are cationizable in the composition according to the disclosure. Non-limiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and mixtures thereof.

In some embodiments, the cationic surfactant is selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof.

In some embodiments, the cationic surfactant comprises cetrimonium chloride, behentrimonium chloride, and mixtures thereof. Behentrimonium Chloride, also described by the technical names that include 1-Docosanaminium, N,N,N-Trimethyl-, Chloride, and N,N,N-Trimethyl-1-Docosanaminium Chloride, is the quaternary ammonium salt that conforms to the formula:

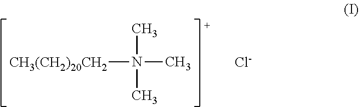

In accordance with some embodiments, the amount of each of the at least one cationic surfactant is from about 0.1 to about 5 wt. %, or about 0.1 to about 4 wt. %, or about 0.2 to about 4 wt. %, or about 0.3 to about 3 wt. %, or about 0.5 to about 3 wt. %, or any suitable combination, sub-combination, range, or sub-range thereof by weight, based on the weight of the composition. One of ordinary skill in the art, however, will appreciate that other ranges are within the scope of the invention. In some particular embodiments, the at least one cationic surfactant, including cationizable surfactants together with an acid neutralizer, is present from about 0.1 to about 5 wt. %, and an acid neutralizer is present from about 0.0.5 to about 1 wt. %, based on the weight of the composition.

Thus, any one of the at least one cationic surfactant is present, by weight, based on the total weight of the composition, from about 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.2, 3.4, 3.5, 3.6, 3.8, 4, 4.2, 4.4, 4.5, 4.6, 4.8, 4.9, or 5 wt.

Non-limiting examples of cationic surfactants include behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quatemium-5), dodecyl dimethyl ethylbenzyl ammonium chloride(Quatemium-14), Quatemium-22, Quatemium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquatemium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The cationic surfactant(s) may also be chosen from optionally polyoxyalkylenated, primary, secondary or tertiary fatty amines, or salts thereof, and quaternary ammonium salts, and mixtures thereof.

In some cases, it is useful to use salts such as chloride salts of the quaternary ammonium compounds.

The fatty amines generally comprise at least one C₈-C₃₀ hydrocarbon-based chain.

A. Examples of quaternary ammonium salts that may especially be mentioned include: those corresponding to the general formula below:

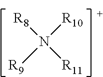

in which the groups R₆ to R₁₁, which may be identical or different, represent a linear or branched, saturated or unsaturated aliphatic group comprising from 1 to 30 carbon atoms, or an aromatic group such as aryl or alkylaryl, at least one of the groups R₈ to R₁₁ denoting a group comprising from 8 to 30 carbon atoms and, in some embodiments, from 12 to 24 carbon atoms. The aliphatic groups may comprise heteroatoms especially such as oxygen, nitrogen, sulfur and halogens. The aliphatic groups are chosen, for example, from C₁-C₃₀ alkyl, C₂-C₃₀ alkenyal, C₁-C₃₀ alkoxy, polyoxy(C₂-C₆)alkylene, C₁-C₃₀ alkylamide, (C₁₂-C₂₂)alkylamido(C₂-C₃₀)alkyl, (C₁₂-C₂₂)alkyl acetate and C₁-C₃₀ hydroxyalkyl groups; X⁻ is an anion chosen from the group of halides, phosphates, acetates, lactates, (C₁-C₄)alkyl sulfates, and (C₁-C₄)alkyl- or (C₁-C₄)alkylarylsulfonates.

Among the quaternary ammonium salts of formula (III), those that are preferred are, on the one hand, tetraalkylammonium salts, for instance dialkyldimethylammonium or alkyltrimethylammonium salts in which the alkyl group contains approximately from 12 to 22 carbon atoms, in particular behenyltrimethylammonium, distearyldimethylammonium, cetyltrimethylammonium or benzyldimethylstearylammonium salts, or, on the other hand, oleocetyldimethylhydroxyethylammonium salts, palmitylamidopropyltrimethylammonium salts, stearamidopropyltrimethylammonium salts and stearamidopropyldimethylcetearylammonium salts.

B. a quaternary ammonium salt of imidazoline, such as, for example, those of formula below:

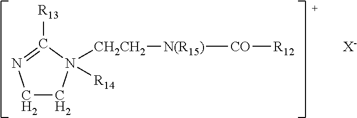

in which R₁₂ represents an alkenyl or alkyl group comprising from 8 to 30 carbon atoms, derived for example from tallow fatty acids, R₁₃ represents a hydrogen atom, a C₁-C₄ alkyl group or an alkyl or alkenyl group comprising from 8 to 30 carbon atoms, R₁₄ represents a C₁-C₄ alkyl group, R₁₅ represents a hydrogen atom or a C₁-C₄ alkyl group, X⁻ is an anion chosen from the group of halides, phosphates, acetates, lactates, alkyl sulfates, alkyl- or alkylaryl-sulfonates in which the alkyl and aryl groups, in some embodiments, comprise, respectively, from 1 to 20 carbon atoms and from 6 to 30 carbon atoms. R₁₂ and R₁₃, in some embodiments, denote a mixture of alkenyl or alkyl groups containing from 12 to 21 carbon atoms, derived for example from tallow fatty acids, R₁₄, in some embodiments, denotes a methyl group, and R₁₅, in some embodiments, denotes a hydrogen atom. Such a product is sold, for example, under the name REWOQUAT W 75 by the company Rewo;

C. a quaternary diammonium or triammonium salt, in particular of formula:

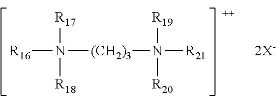

in which R₁₆ denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ)(R₁₇ₐ)(R₁₈ₐ)N—(CH₂)₃, R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms, and X⁻ is an anion chosen from the group of halides, acetates, phosphates, nitrates and methyl sulfates. Such compounds are, for example, Finquat CT-P, sold by the company Finetex (Quatemium 89), and Finquat CT, sold by the company Finetex (Quaternium 75),

D. Cationic/cationizable surfactants, including cationizable surfactants together with an acid neutralizer, for example of the general structure R4-A-R5-B

wherein R4 is a saturated or unsaturated, straight or branched alkyl chain with 8 to 24 C atoms, R5 is a straight or branched alkyl chain with 1 to 4 C atoms, A is selected from:

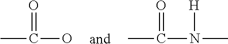

and B is selected from

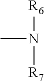

wherein R₆ and R₇ are the same or different is H or an alkyl chain with 1 to 4 C atoms, hydroxyl alkyl chain with 1 to 4 C atoms and di hydroxyl alkyl chain with 2 to 4 C atoms, and

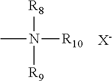

wherein R₈ and R₉ are the same or different, an alkyl chain with 1 to 4 C atoms, hydroxyl alkyl chain with 1 to 4 C atoms and di hydroxyl alkyl chain with 2 to 4 C atoms, R.sub.10 is an alkyl chain with 1 to 4 C atoms, hydroxyl alkyl chain with 1 to 4 C atoms or di hydroxyl alkyl chain with 2 to 4 C atoms.

In some instances, R₄ is saturated or unsaturated, straight or branched alkyl chain with 10 to 24C atoms, in some embodiments, 12 to 22 C atoms and R₅ is straight or branched alkyl group with 1 to 4 C atoms, and A, B, R⁶ to R₁₀ are same as above.

Non-limiting suitable examples are stearyloxypropyl amine, palmityloxypropyl amine, stearyloxypropyldimethyl amine, stearyloxypropyldiethyl amine, stearyloxyethylyldimethyl amine, stearyloxyethyl amine, myristyloxypropyl amine, myristyloxypropyldimethyl amine, palmitamidopropyl amine, palmitamidopropyl methylamine, palmitamidopropyl diethylamine, palmitamidopropyl dibutylamine, palmitamidopropyl buylamine, palmitamidopropyl dipropylamine, palmitamidopropyl propylamine, palmitamidopropyl dihydroxyethylamine, palmitamidopropyl hydroxyethylamine, palmitamidopropyl dihydroxypropylamine, palmitamidopropyl hydroxypropylamine, lauramidopropyl amine, lauramidopropyl methylamine, lauramidopropyl diethylamine, lauramidopropyl dibutylamine, lauramidopropyl buylamine, lauramidopropyl dipropylamine, lauramidopropyl propylamine, lauramidopropyl dihydroxyethylamine, lauramidopropyl hydroxyethylamine, lauramidopropyl dihydroxypropylamine, lauramidopropyl hydroxypropylamine, stearamidopropyl amine, stearamidopropyl dimethylamine, steara midopropyl diethylamine, stearamidopropyldibutylamine, stearamidopropyl butylamine, stearamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, behenamidopropyl amine, behenamidopropyl methylamine, behenamidopropyl diethylamine, behenamidopropyl dibutylamine, behenamidopropyl butylamine, behenamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, dipalmitamidopropyl methylamine, dipalmitamidopropyl ethylamine, dipalmitamidopropyl butylamine, dipalmitamidopropyl propylamine, dipalmitamidopropyl hydroxyethylamine, dipalmitamidopropyl hydroxypropylamine, dilauramidopropyl amine, dilauramidopropyl methylamine, dilauramidopropyl buylamine, dilauramidopropyl hydroxyethylamine, dilauramidopropyl hydroxypropylamine, distearamidopropyl amine, distearamidopropyl methylamine, dibehenamidopropyl propylamine, dibehenamidopropyl hydroxyethylamine, palmitoamidopropyl trimethyl ammonium chloride, stearamidopropyl trimethylammonium chloride, behenamidopropyl tri hydroxyethalmonium chloride, distearylamidopropyl dimethyl ammonium chloride, dicetylamidodihydroxyethyl ammonium chloride, palmitoylpropyl amine, palmitoylpropyl methylamine, palmitoylpropyl diethylamine, palmitoylpropyl dibutylamine, palmitoylpropyl buylamine, palmitoylpropyl dipropylamine, palmitoylpropyl propylamine, palmitoylpropyl dihydroxyethylamine, palmitoylpropyl hydroxyethylamine, palmitoylpropyl dihydroxypropylamine, palmitoylpropyl hydroxypropylamine, myristoylpropyl amine, myristoylpropyl methylamine, myristoylpropyl diethylamine, myristoylpropyl dibutylamine, myristoylpropyl buylamine, myristoylpropyl dipropylamine, myristoylpropyl propylamine, myristoylpropyl dihydroxyethylamine, myristoylpropyl hydroxyethylamine, myristoylpropyl dihydroxypropylamine, myristoylpropyl hydroxypropylamine, stearoylpropyl amine, stearoylpropyl methylamine, stearoylpropyl diethylamine, stearoylpropyl dibutylamine, stearoylpropyl butylamine, stearoylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, behenylpropyl amine, behenylpropyl methylamine, behenylpropyl diethylamine, behenylpropyl dibutylamine, behenylpropyl butylamine, behenylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, dipalmitoylpropyl methylamine, dipalmitoylpropyl ethylamine, dipalmitylpropyl butylamine, dipalmitylpropyl propylamine, dipalmitylpropyl hydroxyethylamine, dipalmitylpropyl hydroxypropylamine, dilauroylpropyl amine, dilauroylpropyl methylamine, dilauroylpropyl buylamine, dilauroylpropyl hydroxyethylamine, dilauroylpropyl hydroxypropylamine, distearylpropyl amine, distearylpropyl methylamine, dibehenylpropyl propylamine, dibehenylpropyl hydroxyethylamine, palmitylpropyl trimethyl ammonium chloride, stearylpropyl trimethylammonium chloride, behenylpropyl tri hydroxyethalmonium chloride, distearylpropyl dimethyl ammonium chloride, dicetyldihydroxyethyl ammonium chloride, dioleoylethylhydroxyethylmonium methosulfate, and dicocoylethylhydroxyethylmonium methosulfate.

Cationizable surfactants or amphiphilic surfactants may be chosen from fatty alkylamines. in some embodiments, fatty dialkylamines. In some cases, the fatty dialkylamines may be fatty dimethylamines. Non-limiting examples include dimethyl lauramine, dimethyl behenamine, dimethyl cocamine, dimethyl myristamine, dimethyl palmitamine, dimethyl stearamine, dimethyl tallowamine, dimethyl soyamine, and mixtures thereof.

Fatty dialkylamines include fatty amidoamine compounds, their salts, and mixtures thereof. Non-limiting examples include oleamidopropyl dimethylamine, linoleamidopropyl dimethylamine, isostearamidopropyl dimethylamine, oleyl hydroxyethyl imidazoline, stearamidopropyl dimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, and palmitamidopropyl dimethylamine.

Non-polymeric, mono-, di-, and/or tri-carboxylic acids may be used to “neutralize” the fatty dialkylamines. In some cases, the one or more non-polymeric, mono-, di-, and/or tri-carboxylic acids include at least one dicarboxylic acid. Non-limiting examples include lactic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, benzoic acid, and mixtures thereof. In particular, lactic acid or tartaric acid or mixtures thereof are useful, especially in combination with fatty dimethylamines such as, for example, stearamidopropyl dimethylamine.

### Fatty Alcohol

In accordance with the disclosure, compositions hereof include at least one fatty alcohol.

The term “fatty alcohol” means an alcohol comprising at least one hydroxyl group (OH), and comprising at least 8 carbon atoms, and which is neither oxyalkylenated (in particular neither oxyethylenated nor oxypropylenated) nor glycerolated. The fatty alcohols can be represented by: R—OH, wherein R denotes a saturated (alkyl) or unsaturated (alkenyl) group, linear or branched, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

The fatty alcohol(s) may be liquid or solid. In some instances, it is preferable that the hair cosmetic compositions include at least one solid fatty alcohol. The solid fatty alcohols that can be used include those that are solid at ambient temperature and at atmospheric pressure (25° C., 780 mmHg), and are insoluble in water, that is to say they have a water solubility of less than 1% by weight, preferably less than 0.5% by weight, at 25° C., 1 atm.

The solid fatty alcohols may be represented by: R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

In particular, it is possible to mention, alone or as a mixture: lauryl alcohol or lauryl alcohol (1-dodecanol); myristic or myristyl alcohol (1-tetradecanol); cetyl alcohol (1-hexadecanol); stearyl alcohol (1-octadecanol); arachidyl alcohol (1-eicosanol); behenyl alcohol (1-docosanol); lignoceryl alcohol (1-tetracosanol); ceryl alcohol (1-hexacosanol); montanyl alcohol (1-octacosanol); myricylic alcohol (1-triacontanol).

Preferably, the solid fatty alcohol is chosen from cetyl alcohol, stearyl alcohol, behenyl alcohol and mixtures thereof such as cetylstearyl or cetearyl alcohol.

The liquid fatty alcohols, in particular those containing C10-C34, preferably have branched carbon chains and/or have one or more, preferably 1 to 3 double bonds. They are preferably branched and/or unsaturated (C═C double bond), and contain from 12 to 40 carbon atoms.

The liquid fatty alcohols may be represented by: R—OH, wherein R denotes a C12-C24 branched alkyl group or an alkenyl group (comprising at least one C12-C24 double bond C═C), R being optionally substituted by one or more hydroxy groups. Preferably, the liquid fatty alcohol is a branched saturated alcohol. Preferably, R does not contain a hydroxyl group. These include oleic alcohol, linoleic alcohol, linolenic alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol and mixtures thereof. Preferably, the liquid fatty alcohol is 2-octyl-1-dodecanol.

In some instances, the hair cosmetic compositions include one or more fatty alcohols selected from decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol and a mixture thereof. In some instances, the hair cosmetic compositions preferably include cetearyl alcohol.

In accordance with the various embodiments, the amount of each of the at least one fatty alcohol is from about 0.5 to about 10 wt. %, or about 2 to about 9 wt. %, or about 3 to about 8 wt. %, or about 3.5 to about 7 wt. %, or about 4 to about 7 wt. %, or about 4.5 to about 6.5 wt. %, or about 5 to about 6 wt. %, or any suitable combination, sub-combination, range, or sub-range thereof by weight, based on the weight of the composition.

In certain preferred embodiments, the total amount of the at least one fatty alcohol is at least 3 wt. %, or at least 3.5 wt. %, or at least 4 wt. % or at least 4.5 wt. % or at least 5 wt. % or at least 5.5 wt. % or at least 6 wt. %, or is in an amount of from about 3 to about 10 wt. %, or about 3.5 to about 10 wt. %, or about 4 to about 9 wt. %, or about 4.5 to about 8.5 wt. %, or about 4.5 to about 8 wt. %, or about 5 to about 7.5 wt. %, or about 5.5 to about 7 wt. %, or about 5.5 to about 7 wt. %, or any suitable combination, sub-combination, range, or sub-range thereof by weight, based on the weight of the composition.

Thus, any one of the at least one fatty alcohol is present, by weight, based on the total weight of the composition, in an amount of from about 3, 3.5, 4, 4.5, 5, 5.5, 5.8, 6, 6.2, 6.5, 7, 7.5 or 8 wt. %.

### Cosmetically Acceptable Solvent

The cosmetically acceptable solvent may be chosen from water, organic solvents, or mixtures thereof.

In an embodiment, the cosmetically acceptable solvent in the compositions of the present invention comprises water.

In an embodiment, the cosmetically acceptable solvent in the compositions of the present invention comprises at least one organic solvent.

In an embodiment, the cosmetically acceptable solvent in the compositions of the present invention comprises water and at least one organic solvent.

### Water

The amount of water in the hair cosmetic compositions may be at least 50 wt. %, or from about 50 to about 95 wt. %, about 50 to about 90 wt. %, about 60 to about 90 wt. %, about 70 to about 88 wt. %, about 75 to about 86 wt. %, based on the weight of the composition, including ranges and sub-ranges there between.

### Organic Solvents

Non-limiting examples of organic solvents include, for example, alcohols (for example, C₁₋₁₅, C₁₋₁₀, or C₁₋₆ alcohols), organic solvents, polyols (polyhydric alcohols and glycols (e.g., glycerin, propylene glycol, butylene glycol, caprylyl glycol, etc.), and a mixture thereof.

Non-limiting examples of organic solvents include monoalcohols and polyols such as ethyl alcohol, isopropyl alcohol, propyl alcohol, benzyl alcohol, and phenylethyl alcohol, or glycerin or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of organic solvents include alkanediols such as 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

In certain embodiments, the at least one organic solvent (non-silicone solvents) includes one or more of propylene glycol, glycerin, ethanol, isopropanol, caprylyl glycol, and benzyl alcohol.

The total amount organic solvent(s) in the hair cosmetic composition, if present, can vary but is typically about 0.01 to about 10 wt. %, based on the total weight of the hair cosmetic composition. In some cases, the total amount of water-soluble solvent(s) is about 0.05 to about 8 wt. %, about 0.1 to about 6 wt. %, about 0.1 to about 5 wt. %, about 0.5 to about 10 wt. %, about 0.5 to about 8 wt. %, about 0.5 to about 6 wt. %, about 0.5 to about 5 wt. %, about 0.5 to about 4 wt. %, or about 0.5 to about 3 wt. %, including all ranges and subranges there between, based on the total weight of the hair cosmetic composition.

In an embodiment, the cosmetically acceptable solvent in the compositions of the present invention comprises water and an organic solvent selected from glycerin, propylene glycol, butylene glycol, caprylyl glycol, isopropyl alcohol, denatured alcohol or ethanol or a mixture thereof. In an embodiment, the at least one organic solvent is in an amount of from about 0.1 to about 6 wt. %, about 0.1 to about 5 wt. %, about 0.5 to about 10 wt. %, about 0.5 to about 8 wt. %, about 0.5 to about 6 wt. %, about 0.5 to about 5 wt. %, about 0.5 to about 4 wt. %, or about 0.5 to about 3 wt. %, including all ranges and subranges there between, based on the total weight of the cosmetic composition.

In an embodiment, the cosmetically acceptable solvent in the compositions of the present invention comprises water and glycerin and/or other higher molecular weight glycol. When present, glycerin and/or other higher molecular weight glycol is in an amount of from about 0.05 to about 2 wt. %, about 0.05 to about 1.5 wt. %, about 0.1 to about 1 wt. %, about 0.1 to about 0.8 wt. %, about 0.1 to about 0.6 wt. %, about 0.1 to about 0.5 wt. %, about 0.1 to about 0.4 wt. %, or about 0.1 to about 0.3 wt. %, including all ranges and subranges there between, based on the total weight of the hair cosmetic composition.

In an embodiment, the cosmetically acceptable solvent in the compositions of the present invention comprises water, propylene glycol, and glycerin.

### Amino Functionalized Silicones

The silicones may be hydrophobic or, in some instances, be functionalized to be hydrophilic. Preferably, the silicones of the hair treatment compositions are amino functionalized silicone. The term “amino-functionalized silicone” means a silicone containing at least one primary amino, secondary amino, tertiary amino and/or quaternary ammonium group. The structure of the amino-functionalized silicone may be linear or branched, cyclic or non-cyclic. The amino functional group may be at any position in the silicone molecule, preferably at the end of the backbone (for example, in the case of amodimethicones) and/or in the side chain.

In some instances, the amino-functionalized silicones are selected from compounds of the following formula:

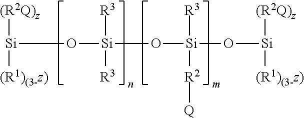

wherein each R¹ is independently selected from a C₁₋₃₀ alkyl group, a C₁₋₃₀ alkoxy group, a C₅₋₃₀ aryl group, a C₆₋₃₀ aralkyl group, a C₆₋₃₀ aralkyloxy group, a C₁₋₃₀ alkaryl group, a C₁₋₃₀ alkoxyaryl group, and a hydroxy group (preferably, each R¹ is independently selected from a C₁₋₃₀ alkyl group, a C₁₋₃₀ alkoxy group and a hydroxy group);

each R² is independently a divalent alkylene radical having one to ten carbon atoms (preferably, R² is a divalent alkylene radical having three to six carbon atoms);

each R³ is independently selected from a C₁₋₃₀ alkyl group, a C₅₋₃₀ aryl group, a C₆₋₃₀ aralkyl group and a C₁₋₃₀ alkaryl group (preferably, each R³ is independently selected from of a C₁₋₃₀ alkyl group);

Q is a monovalent radical selected from —NR⁴₂ and —NR⁴(CH₂)ₓNR⁴₂;

each R⁴ is independently selected from a hydrogen and a C₁₋₄ alkyl group;

x is 2 to 6;

z is 0 or 1;

n is 25 to 3,000 (preferably, 25 to 2,000; more preferably, 25 to 1,000; most preferably 25 to 500); and

m is 0 to 3,000 (preferably, 0 to 2,000; more preferably, 0 to 1,000; most preferably, 0 to 100);

with the proviso that at least 50 mol % of the total number of R¹ and R³ groups are methyl and with the proviso that when m is 0, z is 1.

Preferred R¹ groups include methyl, methoxy, ethyl, ethoxy, propyl, propoxy, isopropyl, isopropoxy, butyl, butoxy, isobutyl, isobutoxy, phenyl, xenyl, benzyl, phenylethyl, tolyl and hydoxy. Preferred R² divalent alkylene radicals include trimethylene, tetramethylene, pentamethylene, —CH₂CH(CH₃)CH₂— and —CH₂CH₂CH(CH₃)CH₂—. Preferred R³ groups include methyl, ethyl, propyl, isopropyl, butyl, isobutyl, phenyl, xenyl, benzyl, phenylethyl and tolyl. Preferred R⁴ groups include methyl, ethyl, propyl, isopropyl, butyl and isobutyl. When z is 0, the amino-functionalized silicone has only pendant amine functional substituents in the polymer chain. When z is 1, the amino-functional silicone may have only terminal amine functional substituents (e.g., m=0) or may have both terminal and pendant amine functional substituents in the polymer chain (e.g., m>0). Preferably, n+m is 50 to 1,000. More preferably, n+m is 50 to 750. Still more preferably, n+m is 50 to 500. Most preferably, n+m is 50 to 250.

In some instances, the amino-functionalized silicones are alkoxylated and/or hydroxylated amino silicones. Suitable alkoxylated and/or hydroxylated amino silicones may be selected from compounds having a structure in accordance with the following

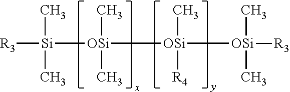

wherein R₃ is hydroxyl or OR₅. R₅ is a C₁ to C₄ alkyl group, R₄ is a group with a structure according to the following formula:

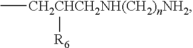

wherein R₆ is a C₁ to C₄ alkyl, n is a 1 to 4, x is the same as “n” described above, and y is the same as “m” described above.

Non-limiting examples of amino-functionalized silicones include bis-hydroxy/methoxy amodimethicones, bis-cetearyl amodimethicone, amodimethicone, bis(C13-15 alkoxy) PG amodimethicones, aminopropyl phenyl trimethicones, aminopropyl dimethicones, bis-amino PEG/PPG-41/3 aminoethyl PG-propyl dimethicones, caprylyl methicones, and a mixture thereof. In some instances, a particularly useful amino-functionalized silicone is bis-hydroxy/methoxy amodimethicone, wherein X is isobutyl and one of the R is OH and the other is OCH₃ in the above structure, also known as “Bis-Hydroxy/Methoxy Amodimethicone” and “3-[(2-aminoethyl)amino]-2-methylpropyl Me, di-Me, [(hydroxydimethylsilyl)oxy]- and [(methoxydimethylsilyl)oxy]-terminated.” Bis-hydroxy/methoxy amodimethicone is commercially available under the tradename DOWSIL AP-8087 FLUID from The Dow Chemical Company.

The silicone of the hair treatment composition may, in some instances, include polydiorganosiloxanes, e.g., polydimethylsiloxanes having the CTFA designation dimethicone. Additional silicones that may be suitable for the hair treatment compositions include (particularly for shampoos and conditioners) polydimethyl siloxanes having hydroxyl end groups, which have the CTFA designation dimethiconol. Silicone gums may, in some instances, be included in the hair treatment compositions, such as those having a slight degree of cross-linking. Non-limiting examples of silicone gums that may, optionally, be included are described in WO 96/31188, which is incorporated herein by reference for all purposes.

The silicone(s) may have a viscosity of at least 10,000 cst, such as at least 50,000 cst, at least 100,000 cst, at least 200,000 cst, at least 400,000 cst, at least 800,000 cst, at least 1,000,000 cst, or at least 2,000,000 cst. Preferably the viscosity does not exceed 109 cst for ease of formulation.

The hair treatment composition may include pre-formed emulsions of silicones, such as emulsions XIAMETER 2-8299 (Dow Corning/Dow Chemical), BELSIL ADM 4000 E (Wacker), DC2-1766, DC2-1784, and microemulsions DC2-1865 and DC2-1870 from Dow Corning, or cross-linked silicone gums, such as DC X2-1787 or DC X2-1391 from Dow Corning.

In an embodiment, the amino functionalized silicone of the compositions of the present invention includes amodimethicone. The amodimethicone may be comercially available as an emulsion comprising amodimethicone, trideceth-6, and cetrimonium chloride under the tradenames XIAMETER 2-8299 or DOWSIL 2-8299 (Dow Corning/Dow Chemical). The amodimethicone may also be available as an emulsion under the tradename, BELSIL ADM 4000 E (Wacker).

In accordance with the various embodiments, the amount of the at least one amino functionalized silicone is from about 0.1 to about 3 wt. %, or preferably about 0.3 to about 2.5 wt. %, or more preferably, about 0.4 to about 2 wt. %, or more preferably, about 0.5 to about 1.5 wt. %, or any suitable combination, sub-combination, range, or sub-range thereof by weight, based on the weight of the composition. One of ordinary skill in the art, however, will appreciate that other ranges are within the scope of the invention.

Thus, any one of the at least one amino functionalized silicone is present, by weight, based on the total weight of the composition, in an amount of from about 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, to about 3 wt. %, including increments and ranges therein and there between.

### Fatty Compounds

The at least one fatty compounds of the compositions of the present invention may be selected from plant-based oils, plant-based butters, plant-based triglycerides, or mixtures thereof.

Non-limiting examples of plant-based or vegetal oils include acai oil, almond oil, aloe vera oil, andiroba oil, annatto oil, avocado oil, babassu oil, borage oil, brazil nut oil, buriti oil, camelina oil, coffee oil, copaiba oil, emu oil, passion fruit oil, almond oil, castor oil, coconut oil, grapeseed oil, jojoba oil, macadamia nut oil, rose hip oil, ajwain oil, angelic root oil, anise oil, aragan oil, asafetida, balsam oil, basil oil, bay oil, bergamot oil, black pepper essential oil, buchu oil, birch oil, camphor, cannabis oil, caraway oil, cardamom seed oil, carrot seed oil, chamomile oil, calamus root oil, cinnamon oil, citronella oil, clary sage, clove leaf oil, coffee, coriander oil, costmary oil, cranberry seed oil, cubeb, cumin oil, cypress, cypriol, curry leaf, davana oil, dill oil, elecampane, eucalyptus oil, fennel seed oil, fenugreek oil, fir, frankincense oil, galangal, geranium oil, ginger oil, goldenrod, grapefruit oil, henna oil, helichrysum, horseradish oil, hyssop, Idaho tansy, jasmine oil, juniper berry oil, lavender oil, lemon oil, lemongrass, marjoram, melaleuca, lemon balm oil, mountain savory, mugwort oil, mustard oil, myrrh oil, myrtle, neem tree oil, neroli, nutmeg, orange oil, oregano oil, orris oil, palo santo, parsley oil, patchouli oil, perilla oil, pennyroyal oil, peppermint oil, petitgrain, pine oil, plum oil, ravensara, red cedar, roman chamomile, rose oil, rosehip oil, rosemary oil, rosewood oil, sandalwood oil, sassafras oil, savory oil, schisandra oil, spikenard, spruce, star anise oil, tangerine, tarragon oil, tea tree oil, thyme oil, tsuga oil, turmeric, valerian, vetiver oil, western red cedar, wintergreen, yarrow oil, ylang-ylang, and zedoary oil, sunflower oil, olive oil, marula oil, corn oil, argan oil, soybean oil, marrow oil, flax oil, sesame oil, hazelnut oil, apricot oil, arara oil, shea butter oil and rapeseed oil.

Suitable fatty compounds for use in the compositions of the present invention can also be selected from plant-based or vegetal butters such as shea butter (Butyrospermum parkii), Karite Nilotica butter (Butyrospermum parkii), galam butter, (Butyrospermum parkii), Borneo butter or fat or tengkawang tallow (Shorea stenoptera), shorea butter, illipe butter, madhuca butter or Bassia madhuca longifolia butter, mowrah butter (Madhuca latifolia), katiau butter (Madhuca mottleyana), phulwara butter (M. butyracea), mango butter (Mangifera indica), murumuru butter (Astrocaryum murumuru), kokum butter (Garcinia indica), ucuuba butter (Virola sebifera), tucuma butter, painya butter (Kpangnan) (Pentadesma butyracea), coffee butter (Coffea arabica), apricot butter (Prunus armeniaca), macadamia butter (Macadamia ternifolia), grapeseed butter (Vitis vinifera), avocado butter (Persea gratissima), olive butter (Olea europaea), sweet almond butter (Prunus amygdalus dulcis), cocoa butter (Theobroma cacao) and sunflower butter.

In a preferred embodiment, the suitable plant-based fatty compound of the present invention is selected from shea butter (Butyrospermum parkii).

The total amount of the plant-based fatty compounds in the composition may vary but is typically from of about 0.1 to about 10 wt %, about 0.1 to about 9 wt. %, or about 0.2 to about 8 wt. %, or about 0.3 to about 7 wt. %, or about 0.4 to about 7 wt. %, or about 0.4 to about 6.5 wt. %, or about 0.5 to about 6 wt. %, or 0.5 to about 5 wt. %, or about 0.1 to about 5 wt %, or about 0.5 to about 4.5 wt. %, or about 0.8 to about 4.2 wt. %, or about 1 to about 4 wt. %, or about 1.2 to about 4 wt. %, or about 1.2 to about 3.5 wt. %, or about 1.5 to about 3 wt %, or about 1 to about 3 wt. %, or about 1.5 to about 2.5 wt %, based on the total weight of the composition, including ranges and sub-ranges there between.

Thus, the fatty compounds is present, by weight, based on the total weight of the composition, in an amount from about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5 to about 5 wt. %, including increments and ranges therein and there between.

### Esters

The esters may be selected from fatty esters, cetyl esters, isopropyl esters, glyceryl (glycerol) esters, dialkyl esters, diesters with octanoic acid and propylene glycol (for example, mixture of the propylene glycol diesters of caprylic and capric acids, INCI: propylene glycol dicaprylate/dicaprate), or mixtures thereof.

Thus, the total amount of esters in the composition, may vary but is typically from about 0.01 to about 6 wt %, such as about 0.05 to about 5.5 wt. %, or about 0.05 to about 5 wt. %, or about 0.05 to about 4.5 wt. %, or about 0.1 to about 4 wt. %, or about 0.2 to about 3.5 wt. %, or about 0.3 to about 3 wt. %, or about 4 to about 2.5 wt. %, or about 0.5 to about 2 wt. %, or about 0.5 to about 1.5 wt. %, based on the total weight of the composition, including ranges and sub-ranges there between.

### Additional Ingredients

The compositions of the present invention may further comprise additional/optional ingredients such as nonionic surfactants.

The nonionic surfactants may be selected from alkoxylated fatty alcohols such as oleth-3, oleth-10, oleth-20, trideceth-5, trideceth-6, trideceth-10, PPG-1 trideceth-6, laureth-12, steareth-20, and combinations thereof,

### Other Components

In one or more embodiments, the hair cosmetic compositions described herein may contain one or more additional ingredients (additives and miscellaneous ingredients). Examples include, but are not limited to amphoteric surfactants, anionic surfactants other than anionic surfactants having at least two carboxylate salt groups, emulsifiers (such as sorbitan esters or polysorbates), thickeners (such as polysaccharide-based thickeners other than cationic guar gums), film formers, other polymers such as cationic polymers, amphoteric polymers, polyquatemium compounds such as polyquaternium-4, polyquatemium-6, polyquatemium-7, polyquatemium-10, polyquatemium-11, polyquatemium-22, polyquatemium-37, polyquatemium-53, polyquatemium-67, etc.), proteins, hydrolyzed proteins, amino acids, fragrance, pH adjusters, chelants, and preservatives.

In an embodiment, the compositions of the present disclosure are in the form of a leave-on product such as a caring product for curly hair (such as combing creams), anti-frizz hair product, or rinse-off or leave-on mask or treatment product.

In an embodiment, the compositions of the present disclosure are in the form of a rinse-off product such as a conditioner product or a mask product or a shampoo-conditioner product (2 in 1).

In an embodiment, the compositions of the present disclosure are in the form of a leave-on product such as a hair or skin treatment product.

In an embodiment, the compositions of the present disclosure are in the form of a leave-on or a rinse-off conditioner.

In an embodiment, the compositions of the present disclosure are in the form of a cream.

In an embodiment, the compositions of the present disclosure are in the form of an emulsion such as an oil-in-water emulsion or a water-in-oil emulsion. In an embodiment, the emulsion is in the form of a cream or lotion.

### pH and Viscosity

The cosmetic composition of the present disclosure has a pH a ranging from about 2.5 to less than about 5, or from about 3 to about 4.8, or from about 3 to about 4.5, or from about 3.2 to about 4.5, or from about 3 to about 4, including ranges and sub-ranges there between.

In various embodiments, the cosmetic composition of the present disclosure has pH of about 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, or 4.9.

In an embodiment, the cosmetic composition of the present disclosure has an initial viscosity (after preparation of the composition) of equal to or greater than 1 Pa·s, or from about 1 to about 100 Pa·s, or about 1 to about 50 Pa·s, or about 1 to about 30 Pa·s, or about 1 to about 20 Pa·s, including ranges and sub-ranges there between, at a shear rate at about or above 10 (1/s) and below 10² 1/s as measured a rheometer (DHR-2, TA instruments, New Castle, Del., USA).

In an embodiment, when the composition is diluted at least 3 times, the viscosity of the composition drops to a viscosity below 10 Pa·s, such as from below 10 to about 1 Pa·s, or at about 9, 8, 7, 6, 5, 4, 3, 2 or 1 Pa·s, at a shear rate above 1 (1/s) and below 10¹ 1/s as measured a rheometer (DHR-2, TA instruments, New Castle, Del., USA).

### Methods

Another aspect of the invention pertains to methods of using the cosmetic compositions described herein. In particular, when the cosmetic compositions are to be applied to hair, i.e., they are hair cosmetic compositions, the methods generally comprise applying any of the cosmetic compositions described to hair. The hair cosmetic compositions may be useful in a variety of settings, and either for chemically treated or pre-alkalized hair or untreated hair. Pre-alkalized hair can include chemically relaxed/straightened hair or chemically dyed or bleached or lightened/highlighted hair, i.e., hair treated with products that result in raising the pH of the hair to an alkaline pH. Use of the compositions of the present invention on hair may include as part of a shampoo, part of a conditioner or as a conditioner, as a pre-treatment, or after cleansing or conditioning or washing the hair, or a treatment for caring for curly hair or as a leave-on or rinse-off mask treatment.

Methods of treating hair according to the disclosure may include applying a hair cosmetic composition of the instant disclosure to the hair (wet, damp, or dry hair), allowing the hair treatment to remain on the hair for a sufficient amount of time, and rinsing the hair cosmetic composition from the hair or allowing the hair treatment to be left on the hair as a leave-on product. The hair cosmetic composition may be applied to the hair before, during, or after other hair cosmetic compositions (e.g., a shampoo, a conditioner, a mask, a cream, a lotion, a gel, etc.).

The hair cosmetic composition may be allowed to remain on the hair for a period of time, for example from about a few seconds (1, 3, 5, or 10 seconds) to about 10, 20, or 30 minutes, or longer such as up to about one hour or up to about two hours or up to about three hours or up to about four hours or up to about five hours or up to about six hours or up to about seven hours or up to about eight hours or up to about 12 hours or overnight.

The hair cosmetic compositions may be useful for treating chemically treated hair.

Described above is the individual application of a hair cosmetic composition or the combined or layered application of a hair cosmetic composition with another composition. In some cases, a hair cosmetic composition is individually applied to the hair and also combined or layered with another composition that is also applied to the hair.

### Kits

The hair cosmetic compositions of the instant disclosure may be incorporated into a kit. For example, the kits may include at least one hair cosmetic composition according to the instant disclosure. The kits may also include one or more hair cosmetic compositions (according the instant disclosure), a shampoo and/or a conditioner and/or a mask and/or other hair treatment of styling product.

Implementation of the present disclosure is provided by way of the following examples. The examples serve to illustrate the technology without being limiting in nature.

### EXAMPLES

The following Examples are intended to be non-restrictive and explanatory only. The ingredient amounts in the compositionstformulas described below are expressed in % by weight, based on the total weight of the composition.

Several formulas were produced having the ingredients as listed in the tables below. The balance of all formulas was water.

### Example I: Compositions

TABLE 1
Formulation Examples

| INGREDIENT | | EX. A | EX. B | EX. C |
|---|---|---|---|---|
| TYPE | INGREDIENT INCI NAME | (invention) | (invention) | (invention) |
| ANIONIC | SODIUM STEAROYL | 1.7 | 0.9 | 1 |
| SURFACTANT | GLUTAMATE(1) | | | |
| CATIONIC | BEHENTRIMONIUM | 1.2 | 1.2 | 2.1 |
| SURFACTANT | CHLORIDE | | | |
| CATIONIC | CETRIMONIUM CHLORIDE | </=0.1 | </=0.1 | </=0.1 |
| SURFACTANT | | | | |
| FATTY ALCOHOL | CETEARYL ALCOHOL | 4.0 | 4.0 | 5.8 |
| SILICONE | AMODIMETHICONE(2) | 1.0 | 1.0 | 1.4 |
| | AND/ORAMODIMETHICONE(3) | | | |
| FATTY | COCOS NUCIFERA | 0.7 | 0.7 | — |
| COMPOUND | (COCONUT) OIL | | | |
| AMINO ACID | ARGININE | 0.2 | — | — |
| NONIONIC | ONE OR MORE OF | </=10 | </=10 | </=10 |
| SURFACTANTS | ALKOXYLATED FATTY | | | |
| | ALCOHOLS, SORBITAN | | | |
| | DERIVATIVES, | | | |
| | POLYETHYLENE GYCOL | | | |
| | ESTER OF FATTY ACIDS (OR | | | |
| | ALKOXYLATED CARBOXYLIC | | | |
| | ACIDS) | | | |
| ORGANIC | ONE OR MORE OF | <2 | <2 | <2 |
| SOLVENTS | GLYCERIN, PROPYLENE | | | |
| | GLYCOL, BUTYLENE | | | |
| | GLYCOL, CAPRYLYL | | | |
| | GLYCOL, ISOPROPYL | | | |
| | ALCOHOL, DENAT. ALCOHOL | | | |
| ADDITIVES OR | ONE OR MORE OF | <5 | <5 | <5 |
| MISCELLANEOUS | PRESERVATIVES, pH | | | |
| INGREDIENTS | ADJUSTERS, CHELANTS, | | | |
| | COLORANTS, SALT, | | | |
| | FRAGRANCE, VITAMINS, | | | |
| | PLANT EXTRACTS, | | | |
| | PROTEINS/AMINO | | | |
| | ACIDS/PROTEIN | | | |
| | HYDROLYSATES | | | |
| | WATER | Q.S. 100 | Q.S. 100 | Q.S. |

(1)commercially available under the tradename AMISOFT HS 11 PF from the company Ajinomoto
(2)commercially available under the tradename DOWSIL AP-8568 AMINO FLUID or DC 2-8566 AMINO FLUID or DOW CORNING AP-8568 AMINO FLUID from the company Dow Corning/DOW Chemical) or under the tradename BELSIL ADM 1370 from the company Wacker
(3)commercially available under the tradename XIAMETER MEM-8299 EMULSION from the company Dow Corning/DOW Chemical) or under the tradename BELSIL ADM 4000 E from the company Wacker

TABLE 2
Comparative Formulation Examples

| INGREDIENT | INGREDIENT | | | | | |
|---|---|---|---|---|---|---|
| TYPE | INCI NAME | EX. D | EX. E | EX. F | EX. G | EX. H |
| ANIONIC | SODIUM STEAROYL | 0.1 | 0.2 | — | — | — |
| SURFACTANT | GLUTAMATE(1) | | | | | |
| CATIONIC | BEHENTRIMONIUM | 1.2 | 2.1 | 1.2 | 1.2 | 2.5 |
| SURFACTANT | CHLORIDE | | | | | |
| CATIONIC | CETRIMONIUM | </=0.1 | </=0.1 | </=0.1 | </=0.1 | </=0.1 |
| SURFACTANT | CHLORIDE | | | | | |
| FATTY ALCOHOL | CETEARYL ALCOHOL | 4.0 | 5.8 | 4.0 | 4.0 | >/==6 |
| SILICONE | AMODIMETHICONE(2) | 1.0 | 1.4 | 1.0 | 1.0 | 2.0 |
| | AND/OR | | | | | |
| | AMODIMETHICONE(3) | | | | | |
| FATTY | COCOS NUCIFERA | 0.7 | — | 0.7 | 0.7 | 0.1 |
| COMPOUND/ | (COCONUT) OIL | | | | | |
| PLANT-BASED | AND/OR | | | | | |
| OIL | ARGANIA SPINOSA | | | | | |
| | KERNEL OIL | | | | | |
| NONIONIC | ONE OR MORE OF | — | 0.1 | — | — | 0.2 |
| SURFACTANT | ALKOXYLATED | | | | | |
| | FATTY ALCOHOLS, | | | | | |
| | SORBITAN | | | | | |
| | DERIVATIVES, | | | | | |
| | POLYETHYLENE | | | | | |
| | GYCOL ESTER OF | | | | | |
| | FATTY ACIDS (OR | | | | | |
| | ALKOXYLATED | | | | | |
| | CARBOXYLIC ACIDS) | | | | | |
| AMINO ACID | ARGININE | — | — | — | 0.2 | — |
| ANIONIC | PALMITIC ACID | — | — | — | 0.5 | — |
| SURFACTANT/ | STEARIC ACID | — | — | — | 0.6 | — |
| FATTY ACID | MYRISTIC ACID | — | — | — | 0.0 | — |
| ORGANIC | ONE OR MORE OF | <2 | <2 | <2 | <2 | <2 |
| SOLVENTS | GLYCERIN, | | | | | |
| | PROPYLENE | | | | | |
| | GLYCOL, BUTYLENE | | | | | |
| | GLYCOL, CAPRYLYL | | | | | |
| | GLYCOL, ISOPROPYL | | | | | |
| | ALCOHOL, DENAT. | | | | | |
| | ALCOHOL | | | | | |
| ADDITIVES OR | ONE OR MORE OF | <5 | <5 | <5 | <5 | <5 |
| MISCELLANEOUS | PRESERVATIVES, | | | | | |
| INGREDIENTS | pH ADJUSTERS, | | | | | |
| | CHELANTS, NONIONIC | | | | | |
| | SURFACTANTS, | | | | | |
| | COLORANTS, SALT, | | | | | |
| | FRAGRANCE, | | | | | |
| | VITAMINS, PLANT | | | | | |
| | EXTRACTS, | | | | | |
| | PROTEINS/AMINO | | | | | |
| | ACIDS/PROTEIN | | | | | |
| | HYDROLYSATES | | | | | |
| | WATER | Q.S. 100 | Q.S. 100 | Q.S. 100 | Q.S. 100 | Q.S. 100 |

### Process of Making the Invention Compositions:

The invention examples were prepared according to the following general process: The formulation was prepared by:
heating water to about 70° C.,
then at about 70° C., adding benzoic acid, arginine, cetearyl alcohol, behentrimonium chloride, sodium stearoyl glutamate, amodimethicone and fatty oil, with a waiting time between each compound until the added compound has liquefied before adding the next,
once all compounds have been dispersed, cooling to about 50° C.,
adding other ingredients and optional additives (preservatives, organic solvent, surfactants, perfume and dyes etc.),
then continuing to cool down to room temperature, and
adjusting pH, if needed.

The pH of the inventive formulas made according to the procedure above ranged from about 2.5 to less than 5. The stability of the inventive formulas was also checked to show that after 2 months at 45° C. and 4° C., respectively, there was no phase separation or precipitation, even when they contained a combination of a cationic compound (cationic surfactant) and an anionic compound/surfactant.

### Example II Viscosity, PH, and Dilution Studies

The following table shows the changes in properties of the inventive formulations such as texture and viscosities when their pH was changed and/or when the formulations were diluted with water. The viscosities of the formulations were measured using a rheometer (DHR-2, TA instruments, New Castle, Del., USA) and 40 mm parallel plate geometry. The 1 mm gap between the parallel plates was chosen. All tests were conducted at 25° C. and atmospheric pressure. The sample was subjected to shear ramp starting from 0.1 1/s to 1000 1/s within a 300 second period. The time interval between data points is 1 s. The shear stress response was recorded for every data point.

The pH of the formulations was changed or adjusted by adding sodium hydroxide solution or other appropriate pH modifiers.

The textures of the formulations were evaluated using a polarized microscope.

TABLE 3

| | | | | EX. D | EX. E | EX. F | EX. G | EX. H |
|---|---|---|---|---|---|---|---|---|
| | EX. A | EX. B | EX. C | (compara- | (compara- | (compara- | (compara- | (compara- |
| | (invention) | (invention) | (invention) | tive) | tive) | tive) | tive) | tive) |
| MOLAR RATIO | 0.8 or | 1.6 or | 2.26 or | 12.3 | 11.4 | — | 0.83* | — |
| OF CATIONIC | 1:1.26 | 1:0.63 | 1:0.44 | | | | | |
| SURFACTANT | (<1) | (>1) | (>1) | | | | | |
| TO ANIONIC | | | | | | | | |
| SURFACTANT | | | | | | | | |
| pH of | 4.8 | 3.4 | 3.3 | 3.2 | 3.0 | 3.4 | 3.8 | 5.1 |
| composition (non- | | | | | | | | |
| diluted) | | | | | | | | |
| Range of | 16.8-0.07 | 10.3-0.11 | 1.8-0.12 | 5.8-0.13 | 2.1-0.073 | 5.1-0.15 | 4.2-0.34 | 4.8-0.18 |
| viscosity (Pa · s) | | | | | | | | |
| over a shear rate | | | | | | | | |
| of 10 to 1 × 10³ | | | | | | | | |
| 1/s | | | | | | | | |
| Texture of | lamellar | lamellar | lamellar | lamellar | lamellar | lamellar | lamellar | lamellar |
| composition | structure | structure | structure | structure | structure | structure | structure | structure |
| | and creamy | and creamy | and creamy | and creamy | and creamy | and creamy | and creamy | and creamy |

pH change

| | | | | | | | | | |
|---|---|---|---|---|---|---|---|---|---|
| Change in the pH | decreased | increased | increased | Increased | increased | Increased | increased | increased | decreased |
| of the | to 3.5 | to 5.4 | to 5.5 | to 5.7 | to 5.3 | to 6.2 | to 5.8 | to 7.7 | to 3.6 |
| composition | | | | | | | | | |

| | | | | | | | | |
|---|---|---|---|---|---|---|---|---|
| Viscosity and | viscosity | viscosity | Viscosity | change in | change in | change in | change in | change in |
| texture of | significantly | significantly | significantly | viscosity | viscosity | viscosity | viscosity | viscosity |
| composition after | decreased | decreased | decreased | was very | was very | was very | was very | was very |
| change in pH | and | and | and | small and | small and | small and | small and | small and |
| | structure | structure | structure | lamellar | lamellar | lamellar | lamellar | lamellar |
| | broke down, | broke down; | broke down, | structure | structure | structure | structure | structure |
| | phase | phase | phase | remained | remained | remained | remained | remained |
| | separation | separation/ | separation | intact | intact | intact | intact | intact |
| | | precipitation | | | | | | |

** 3x Dilution with Extraneous Water

| | | | | | | | | | |
|---|---|---|---|---|---|---|---|---|---|
| Range of | 0.33-0.012 | 0.25-0.013 | 0.63-0.039 | 4.9-0.11 | 1.96-0.066 | 3.9-0.13 | 7.0-0.16 | 5.0-0.12 | 5.1-0.22 |
| viscosity (Pa · s) | | | | | | | | | |
| over a shear rate | | | | | | | | | |
| of 10 to 1 × 10³ | | | | | | | | | |
| 1/s after change | | | | | | | | | |
| in pH | | | | | | | | | |

| | | | | | | | | |
|---|---|---|---|---|---|---|---|---|
| Viscosity after 3x | Viscosity | Viscosity | Viscosity | Viscosity | change in | Viscosity | Viscosity | change in |
| dilution | decreased | decreased | decreased | decreased | viscosity | decreased | decreased | viscosity |
| | significantly | significantly | significantly | significantly | was very | significantly | significantly | was very |
| | | | | | small | | | small |
| Range of | 0.74-0.013 | 0.48-0.036 | 0.48-0.022 | 0.16-0.028 | 1.85-0.059 | 0.42-0.069 | 0.32-0.022 | 4.3-0.07 |
| viscosity (Pa · s) | | | | | | | | |
| over a shear rate | | | | | | | | |
| of 10 to 1 × 10³ | | | | | | | | |
| 1/s after 3x | | | | | | | | |
| dilution | | | | | | | | |

*
** extraneous water (or added water) refers to water that is not part of the formulation when it is first prepared

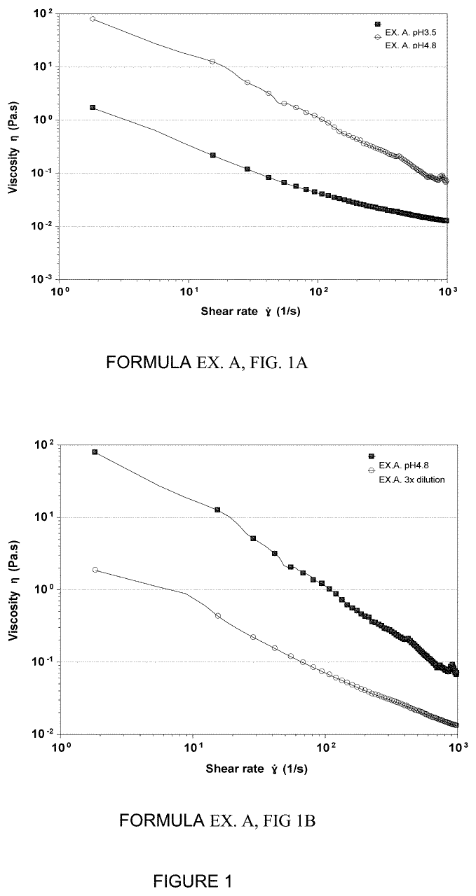

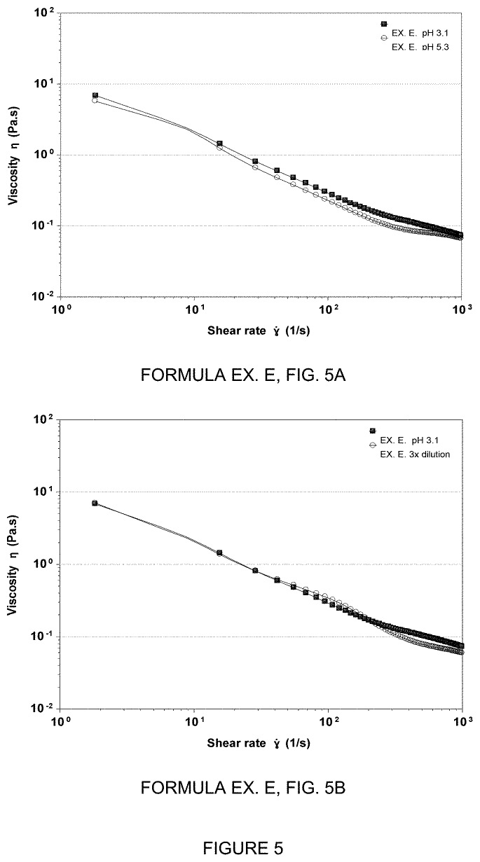

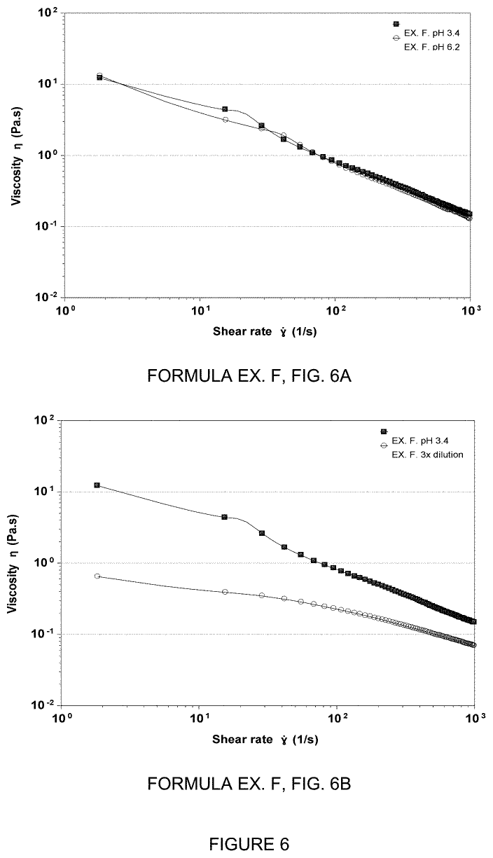

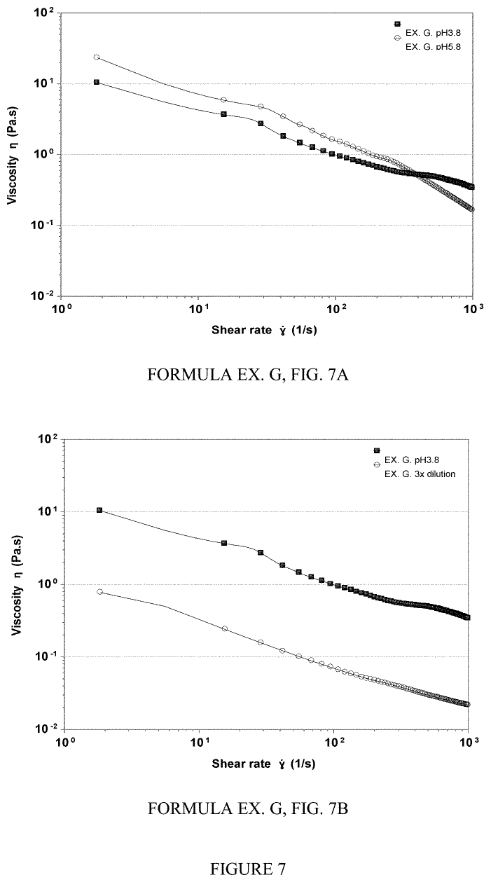

The data in Table 3 can be visualized in FIGS. 1 to 8. The inventive compositions Ex. A to C each had a pH below 5. Ex. A composition employed a molar ratio of cationic surfactants to anionic surfactants of less than 1 and a pH of above 4. Ex. B and C compositions each employed a molar ratio of cationic surfactants to anionic surfactants of greater than 1 (from greater to 1 to equal to or less than 10) and a pH of below 4.

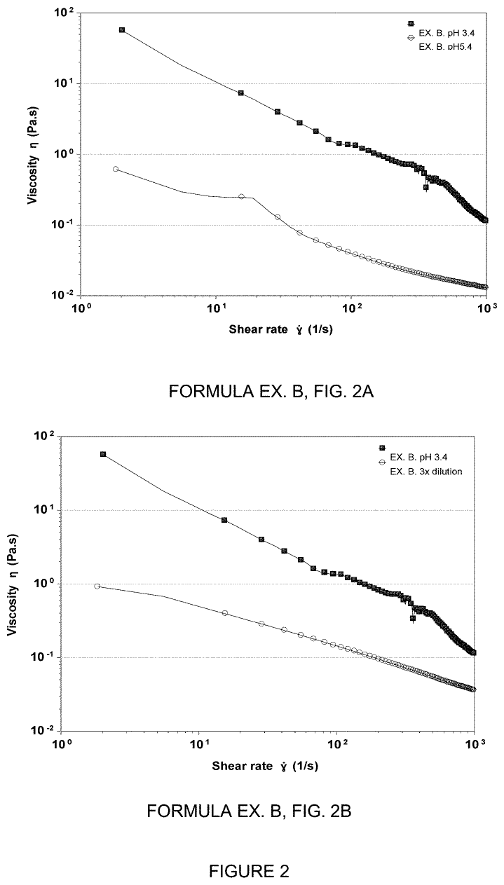

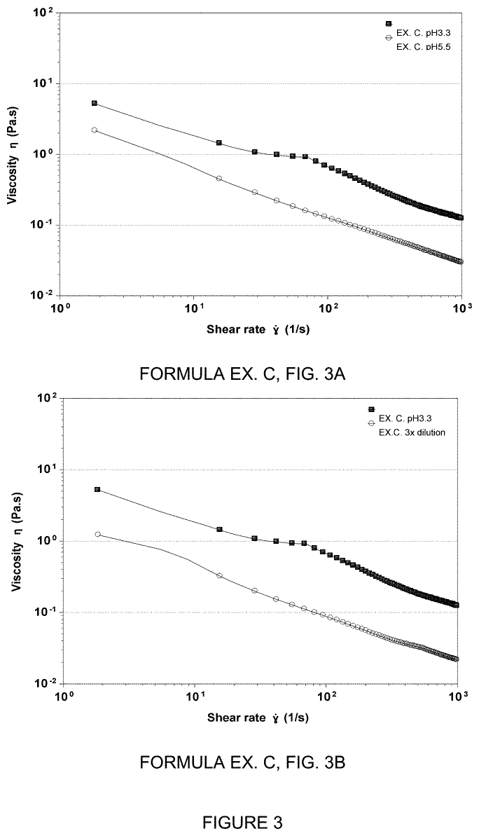

FIG. 1 shows the significant decrease in viscosity of the inventive composition Ex. A when the pH was decreased to lower than 4, i.e., from 4.8 to 3.5 (FIG. 1A) or when the composition was diluted 3 times (FIG. 1B). FIG. 2 shows the significant decrease in viscosity of the inventive composition Ex. B when the pH was increased to higher than 5, i.e., from 3.4 to 5.4 (FIG. 2A) or when the composition was diluted 3 times (FIG. 2B). FIG. 3 shows the significant decrease in viscosity of the inventive composition Ex. C when the pH was increased to higher than 5, i.e., from 3.3 to 5.5 (FIG. 3A) or when the composition was diluted 3 times (FIG. 38). The decrease in viscosity allows for easier spreadability and application on a surface such the surface of hair fibers which results in better and homogeneous deposition of active agents and/or conditioning agents on the surface. The viscosity drop also allows for easier and faster removal of the composition from the surface (fast wash or fast rinse effect). The cleansing or rinsing process therefore requires less water. These effects are particularly useful for chemically treated hair (or pre-alkalized hair) which have previously been treated with hair dyes or relaxer products that are typically alkaline and which typically have residual alkalinity. The treatment of pre-alkalized hair with the inventive compositions result in a change or increase in pH which allows the viscosity to drop while the composition is being spread on the pre-alkalized hair.

At the same time, the comparative compositions Ex. E, D, F, G, and H were tested and compared to Ex. A, B, and C. Ex. D and E compositions had a pH of below 5, contained very little anionic surfactants (0.1 to 0.2 wt %), and had a molar ratio of cationic surfactants to anionic surfactants of greater than 10 (i.e., at 12.3 and 11.4, respectively). Ex. F and H compositions had a pH of below 5 and zero amount of anionic surfactants. Ex. G composition had a pH above 5 and had a molar ratio of cationic surfactants to anionic surfactants (fatty acid with only one carboxylic group) of less than 1 (i.e., at 0.83).

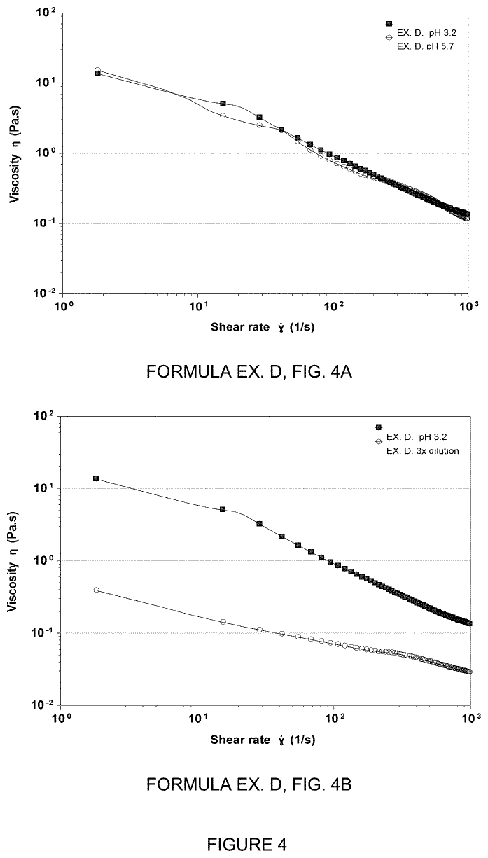

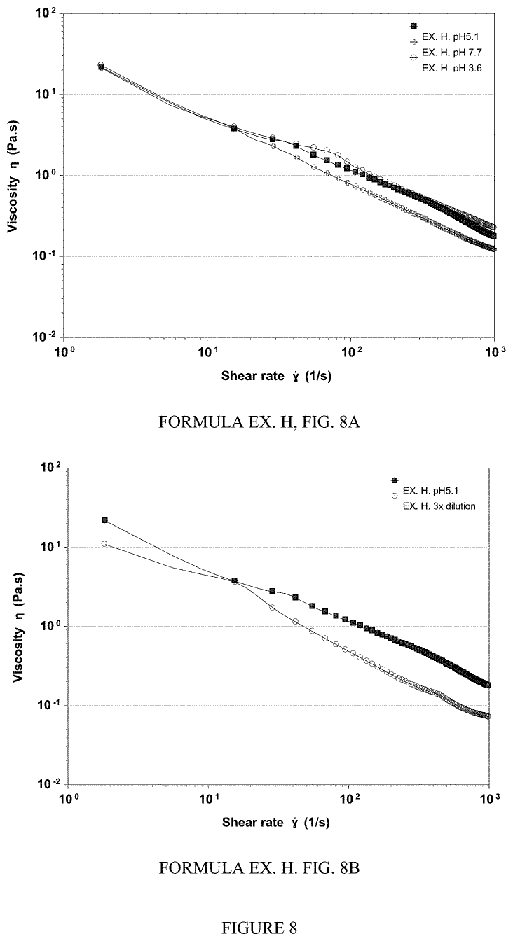

When the pH was increased to higher than 5 for each of the comparative compositions Ex. E, D, F, G, and H they all demonstrated only very small changes in viscosities (FIGS. 4A, 5A, 6A, 7A, and 8A). In addition, when the pH of Ex. H composition was decreased from 5.1 to below 5 (i.e., 3.6), there was also very little change in viscosity (FIG. 8A). These results show that these compositions are not responsive to pH changes. Thus, even when applied on pre-alkalized hair (e.g., hair treated with hair dyes or relaxer products that are typically alkaline and which typically have residual alkalinity) or even when contacted with either an alkaline or acidic composition, it can be anticipated that the application and spreadability of Ex. D, E, F, G, and H compositions on hair would not change or improve. The results also indicate that there would be no change or impact on the efficiency or speed of removability or rinsability of these compositions from keratinous substrates treated with such compositions even when exposed to significant changes in pH.

When the comparative compositions were diluted with water three times, Ex. D, F, and G demonstrated significant decreases in viscosity, while Ex. E and H showed very little changes in viscosity (FIGS. 4B, 5B, 6B, 7B, and 8B). Thus, Ex. D, F and G were responsive with respect to viscosity changes only during dilution of the compositions with water (but not pH responsive). On the other hand, Ex. E and H were not responsive to either pH changes to or dilution of the compositions.

As a conclusion, the inventive compositions were found to be responsive to changes in pH or dilution with water that results in significant changes in viscosity of the compositions.

When the molar ratio of the cationic surfactant to the anionic surfactant having a least two carboxylate salt groups in the composition is greater than about 1 and less than 10 and the pH of the composition is increased either by applying the composition on pre-alkalized hair or combining it with an alkaline or basic composition (such as a hair dye composition), the viscosity of the composition decreases significantly. When the molar ratio of the cationic surfactant to the anionic surfactant having a least two carboxylate salt groups in the composition is less than about 1 and the pH of the composition is decreased, the viscosity of the composition also decreases significantly. In both instances, the decrease in viscosity allows for improving the spreadability and ease of application of the composition on hair.

When the molar ratio of the cationic surfactant to the anionic surfactant having a least two carboxylate salt groups in the composition is greater than about 1 and less than 10 and the composition is diluted with water (such as in hair cleansing or conditioning activities), the viscosity of the composition decreases significantly. This also allows for improving the spreadability and ease of application of the composition on hair.

At the same time, the compositions become easier to be removed or rinsed/washed off from the hair (fast wash or fast rinse effect).

The foregoing description illustrates and describes the invention. The disclosure shows and describes only the preferred embodiments but it should be understood that the invention is capable to use in various other combinations, modifications, and environments and is capable of changes or modifications within the scope of the inventive concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the claimed invention.

The terms “a,” “an,” and “the” are understood to encompass the plural as well as the singular.

Thus, the term “a mixture thereof” also relates to “mixtures thereof.” Throughout the disclosure, if the term “a mixture thereof” is used, following a list of elements as shown in the following example where letters A-F represent the elements: “one or more elements selected from the group consisting of A, B, C, D, E, F, or mixtures thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counterion such as an alkali metal, alkaline earth metal, or ammonium counterion. This list of counterions, however, is non-limiting.

The expression “one or more” means “at least one” and thus includes individual components as well as mixtures/combinations.

The term “plurality” means “more than one” or “two or more.”

The term “lasting” or “long lasting” or “durable” as used herein means that the cosmetic attribute or effect was observed up to about 30 minutes or up to about one hour or up to about two hours, or up to about three hours or up to about four hours or up to about five hours or up to about six hours or up to about seven hours or up to about eight hours or up to about 12 hours or after an overnight period from the time the composition of the present disclosure was applied to hair on the head of a person and the hair was subjected to changes in the hair shape or style or configuration, and/or to disturbance of the hair fibers and/or to movement of the hair fibers as a result of sleeping or lying down and resting the head on a bed or other surface or the back part of a seat and/or as a result of wearing a head cap or cover for at least 30 minutes up to several hours or overnight.

Some of the various categories of components identified for the hair-treatment compositions may overlap. In such cases where overlap may exist and the composition/product includes two overlapping components (or more than two overlapping components), an overlapping component does not represent more than one component. For example, a fatty acid may be defined as both a “fatty compound” and a “surfactant/emulsifier.” If a particular composition/product includes both a fatty compound component and an emulsifier component, a single fatty acid can serve as only a fatty compound or a surfactant/emulsifier (a single fatty acid does not serve as both the fatty compound and the surfactant/emulsifier).

All percentages, parts and ratios herein are based upon the total weight of the compositions of the present invention, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub-ranges between, the given ranges. Thus, a range from 1-5, includes specifically points 1, 2, 3, 4 and 5, as well as sub-ranges such as 2-5, 3-5, 2-3, 2-4, 1-4, etc.; and points of 1, 2, 3, 4, and 5 includes ranges and sub-ranges of 1-5, 2-5, 3-5, 2-3, 2-4, 1-4, etc.

Other than in the operating examples, or where otherwise indicated, all numbers expressing quantities of ingredients and/or reaction conditions are understood to be modified by “about,” whether or not expressly stated. Additionally, all numbers are intended to represent exact figures as additional embodiments, whether or not modified by “about.” For example, “an amount of about 1%” includes an amount of exactly 1%. As a further example, “an amount of 1%” includes an amount of about 1%. The term “about” is generally understood to encompass a range of +/−10% from the stated number, and is intended to cover amounts of +/−1%, 2%, 3%, 4%, 5%, 6%, 7%, 8%, 9%, and 10%.

The term “surfactants” includes salts of the surfactants even if not explicitly stated. In other words, whenever the disclosure refers to a surfactant, it is intended that salts of the surfactant are also encompassed to the extent such salts exist, even though the specification may not specifically refer to a salt (or may not refer to a salt in every instance throughout the disclosure), for example, by using language such as “a salt thereof” or “salts thereof.” Sodium and potassium are common cations that form salts with surfactants. However, additional cations such as ammonium ions, or alkanolammonium ions such as monoethanolammonium or triethanolammonium ions, may also form salts of surfactants.

The term “substantially free” or “essentially free” as used herein means the specific material may be present in small amounts that do not materially affect the basic and novel characteristics of the claimed invention. For instance, there may be less than 2% by weight of a specific material added to a composition, based on the total weight of the compositions (provided that an amount of less than 2% by weight does not materially affect the basic and novel characteristics of the claimed invention. Similarly, the compositions may include less than 2 wt %, less than 1.5 wt %, less than 1 wt %, less than 0.5 wt %, less than 0.3 wt %, less than 0.1 wt %, less than 0.05 wt %, or less than 0.01 wt %, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure.

The term “substantially free” or “essentially free” as used herein may also mean that the specific material is not added to the composition but may still be present in a raw material that is included in the composition.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A cosmetic composition comprising:
(a) at least one anionic surfactant having at least 2 carboxylate salt groups;
(b) at least one cationic surfactant, including a cationizable surfactant;
(c) at least one fatty alcohol; and
(d) at least one cosmetically acceptable solvent;
wherein the mole ratio of the at least one cationic surfactant to the at least one at least one anionic surfactant ranges from about 5:1 to about 1:5.

2. The cosmetic composition of claim 1, wherein the at least one anionic surfactant having at least 2 carboxylate salt groups is represented by the formula (I): 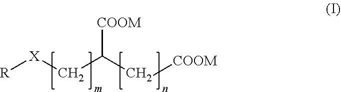
wherein:
R represents a saturated or unsaturated, linear or branched hydrocarbon group having 8 to 30 carbon atoms,
X is —O— or an ester group, —COO—, or amide group, —CONY wherein Y is hydrogen or an alkyl group,
m is 0 or 1 or 2,
n is an integer of 0 to 4,
M is hydrogen or a salt-forming cation wherein COO is the counter-anion.

5. The cosmetic composition of claim 1, wherein the at least one anionic surfactant having at least two carboxylate salt groups is selected from sodium stearoyl glutamate, sodium lauroyl aspartate, disodium stearoyl glutamate, disodium lauroyl aspartate, sodium cocoyl glutamate, disodium cocoyl glutamate, sodium lauroyl glutamate, or a mixture thereof.

6. The cosmetic composition of claim 1, wherein the at least one anionic surfactant having at least two carboxylate salt groups is present in an amount of from about 0.5 to about 5 wt. %, based on the total weight of the hair cosmetic composition.

7. The cosmetic composition of claim 1, wherein the at least one cationic surfactant is selected from:
quaternary ammonium salts corresponding to the general formula below: 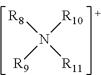
in which the groups R₈ to R₁₁, which may be identical or different, represent a linear or branched, saturated or unsaturated aliphatic group comprising from 1 to 30 carbon atoms, or an aromatic group such as aryl or alkylaryl, at least one of the groups R₈ to R₁₁ denoting a group comprising from 8 to 30 carbon atoms;
a quaternary ammonium salt of imidazoline;
a quaternary diammonium or triammonium salt, in particular of formula: 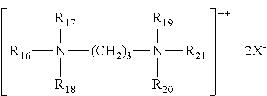
in which R₁₆ denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ)(R₁₇ₐ)(R₁₈ₐ)N—(CH₂)₃, R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms, and X⁻ is an anion chosen from the group of halides, acetates, phosphates, nitrates and methyl sulfates; and
cationizable surfactants, including cationizable surfactants together with an acid neutralizer selected from compounds of the general structure R₄-A-R₅—B,
wherein R₄ is a saturated or unsaturated, straight or branched alkyl chain with 8 to 24 C atoms, R₅ is a straight or branched alkyl chain with 1 to 4 C atoms, A is selected from: 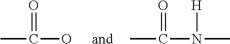
and B is selected from 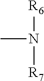
wherein R₆ and R₇ are the same or different is H or an alkyl chain with 1 to 4 C atoms, hydroxyl alkyl chain with 1 to 4 C atoms and di hydroxyl alkyl chain with 2 to 4 C atoms, and 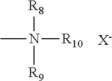
wherein R₈ and R₉ are the same or different, an alkyl chain with 1 to 4 C atoms, hydroxyl alkyl chain with 1 to 4 C atoms and di hydroxyl alkyl chain with 2 to 4 C atoms, R₁₀ is an alkyl chain with 1 to 4 C atoms, hydroxyl alkyl chain with 1 to 4 C atoms or di hydroxyl alkyl chain with 2 to 4 C atoms,
or mixtures thereof.

8. The cosmetic composition of claim 1, wherein the at least one cationic surfactant is selected from quaternary diammonium or triammonium salts, in particular of formula: 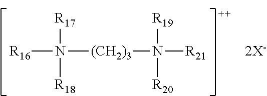
in which R₁₆ denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ)(R₁₇ₐ)(R₁₈ₐ)N—(CH₂)₃, R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms, and X⁻ is an anion chosen from the group of halides, acetates, phosphates, nitrates and methyl sulfates.

9. The cosmetic composition of claim 1, wherein the at least one cationic surfactant is selected from cationizable surfactants, including cationizable surfactants together with an acid neutralizer selected from compounds of the general structure R4-A-R5-B,
wherein R₄ is a saturated or unsaturated, straight or branched alkyl chain with 8 to 24 C atoms, R₅ is a straight or branched alkyl chain with 1 to 4 C atoms, A is selected from: 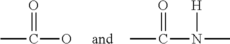
and B is selected from 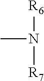
wherein R₆ and R₇ are the same or different is H or an alkyl chain with 1 to 4 C atoms, hydroxyl alkyl chain with 1 to 4 C atoms and di hydroxyl alkyl chain with 2 to 4 C atoms, and 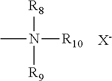
wherein R₈ and R₉ are the same or different, an alkyl chain with 1 to 4 C atoms, hydroxyl alkyl chain with 1 to 4 C atoms and di hydroxyl alkyl chain with 2 to 4 C atoms, R₁₀ is an alkyl chain with 1 to 4 C atoms, hydroxyl alkyl chain with 1 to 4 C atoms or di hydroxyl alkyl chain with 2 to 4 C atoms.

10. The cosmetic composition of claim 1, wherein the at least one cationic surfactant is selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, or mixtures thereof.

11. The cosmetic composition of claim 1, wherein the at least one cationic surfactant is present in an amount of from about 0.1 to about 5 wt. %, based on the total weight of the hair cosmetic composition.

12. The cosmetic composition of claim 1, wherein the at least one fatty alcohol is selected from cetyl alcohol, stearyl alcohol, cetearyl alcohol (combination of cetyl alcohol and stearyl alcohol), behenyl alcohol, lauryl alcohol (1-dodecanol); myristic or myristyl alcohol (1-tetradecanol), arachidyl alcohol (1-eicosanol), lignoceryl alcohol (1-tetracosanol); ceryl alcohol (1-hexacosanol); montanyl alcohol (1-octacosanol); myricylic alcohol (1-triacontanol), decyl alcohol, undecyl alcohol, and a mixture thereof.

13. The cosmetic composition of claim 1, wherein the at least one fatty alcohol is present in an amount of from about 0.5 to about 10 wt. %, based on the total weight of the hair cosmetic composition.

14. The cosmetic composition of claim 1, wherein the at least one cosmetically acceptable solvent is selected from water, organic solvents, and mixtures thereof.

15. The cosmetic composition of claim 1, wherein the molar ratio of the at least one cationic surfactant to the at least one anionic surfactant is greater than 1.

16. The cosmetic composition of claim 14, wherein the molar ratio of the at least one cationic surfactant to the at least one anionic surfactant ranges from about 1:0.9 to about 1:0.2.

17. The cosmetic composition of claim 1, wherein the molar ratio of the at least one cationic surfactant to the at least one anionic surfactant is less than 1.

18. The cosmetic composition of claim 16, wherein the molar ratio of the at least one cationic surfactant to the at least one anionic surfactant ranges from about 1:2 to about 1:1.1.

19. The cosmetic composition of claim 1, wherein the cosmetic composition has a pH ranging from about 2.5 to less than about 5.

20. The cosmetic composition of claim 1, wherein the composition has an initial viscosity (after preparation of the composition) of equal to or greater than 1 Pa·s, at a shear rate above 10 (1/s) and below 10² 1/s as measured by a rheometer (DHR-2, TA instruments, New Castle, Del., USA).

21. The cosmetic composition of claim 1, wherein the molar ratio of the at least one cationic surfactant to the at least one anionic surfactant is greater than 1 and the pH of the composition is less than 5.

22. The cosmetic composition of claim 1, wherein the molar ratio of the at least one cationic surfactant to at least one anionic surfactant having at least two carboxylate salt groups is less than 1 and the pH of the composition is less than 5.

23. The cosmetic composition of claim 1, wherein the composition further comprises at least one silicone including at least one amino functionalized silicone selected from amodimethicone, bis-hydroxy/methoxy amodimethicones, bis-cetearyl amodimethicone, amodimethicone, bis(C13-15 alkoxy) PG amodimethicones, aminopropyl phenyl trimethicones, aminopropyl dimethicones, bis-amino PEG/PPG-41/3 aminoethyl PG-propyl dimethicones, caprylyl methicones, or mixtures thereof; and wherein the at least one silicone is present in an amount of from about 0.1 to about 5 wt. %, based on the total weight of the hair cosmetic composition.

24. The cosmetic composition of claim 1, wherein the composition further comprises at least one nonionic surfactant selected from alkoxylated fatty alcohols, sorbitan derivatives, polyethylene glycol ester of fatty acids (or alkoxylated carboxylic acids), or a mixture thereof and wherein the at least one nonionic surfactant is present in an amount of from about 0.05 to about 10 wt. %, based on the total weight of the cosmetic composition.

25. The cosmetic composition of claim 1, wherein the composition further comprises at least one fatty compound is selected from plant-based oils, hydrocarbon oils, esters, or a mixture thereof; and wherein the at least one fatty compound is present in an amount of from about 0.1 to about 10 wt. %, based on the total weight of the hair cosmetic composition.

26. A cosmetic composition comprising:
(a) from about 0.5 to about 5 wt. % of at least one anionic surfactant having at least two carboxylate salt groups;
(b) from about 0.2 to about 5 wt. % of at least one cationic surfactant, including a cationizable surfactant;
(c) from about 2 to about 7 wt. % of at least one fatty alcohol selected;
(d) at least one cosmetically acceptable solvent;
(e) optionally, from about 0.1 to about 2 wt. % of at least one silicone;
(f) optionally, from about 0.05 to about 2 wt. % of at least one nonionic surfactant; and
(g) optionally, from about 0.1 to about 5 wt. % of at least one fatty compound selected from plant-based oils, hydrocarbon oils, esters, fatty acids, or a mixture thereof; and
wherein the molar ratio of the at least one cationic surfactant to the at least one anionic surfactant ranges from about 4:1 to about 1:4;
all weights being based on the total weight of the cosmetic composition.

27. A method of treating hair, the method comprising applying onto hair, the cosmetic composition of claim 1.

28. A method of treating hair, the method comprising a step of applying onto hair, a cosmetic composition comprising:
(a) at least one anionic surfactant having at least two carboxylate salt groups;
(b) at least one cationic surfactant, including a cationizable surfactant;
(c) at least one fatty alcohol;
(d) at least one cosmetically acceptable solvent;
(e) optionally, at least one silicone;
(f) optionally, at least one fatty compound selected from non-silicone oils, esters, fatty acids, or a mixture thereof; and
wherein the molar ratio of the at least one cationic surfactant to the at least one anionic surfactant ranges from about 5:1 to about 1:5.

29. The method of claim 28, wherein the method further comprises a step of pre-alkalizing hair preceding the step of applying the cosmetic composition onto hair.

30. The method of claim 28, wherein when the keratinous substrate is hair, the hair has been pre-alkalized or previously contacted with an alkaline treatment.

31. The method of claim 28, wherein the method further comprises a step of rinsing the hair with water following the step of applying the cosmetic composition onto hair.

32. The method of claim 28, wherein when the mole ratio of the at least one cationic surfactant to the at least one anionic surfactant is greater than 1, the pH of the composition increases during the rinsing step.

33. The method of claim 32, wherein the method imparts a fast wash or a fast rinse effect to the rinsing step.

34. The method of claim 28, wherein the method includes imparting to hair one or more of:
conditioning benefits;
hair repair benefits;
frizz control;
manageability;
curl definition;
sleek look;
smoothness; or
softness.

35. A method of improving or increasing the removability of a cosmetic composition from hair, the method comprising combining;
(a) from about 0.5 to about 5 wt. % of at least one anionic surfactant having at least two carboxylate salt groups;
(b) from about 0.2 to about 5 wt. % of at least one cationic surfactant, including a cationizable surfactant;
(c) from about 2 to about 7 wt. % of at least one fatty alcohol selected;
(d) at least one cosmetically acceptable solvent;
(e) optionally, at least one silicone;
(f) optionally, at least one nonionic surfactant; and
(g) optionally, at least one fatty compound selected from plant-based oils, hydrocarbon oils, esters, fatty acids, or a mixture thereof; and
in order to form, a cosmetic composition;
all weights being based on the total weight of the cosmetic composition;
wherein the molar ratio of the at least one cationic surfactant to the at least one anionic surfactant ranges from about 5:1 to about 1:5.
